(19) United States
(12) Patent Application Publication
(10) Pub. No.: US 2026/0269065 A1
(43) Pub. Date: Sep. 10, 2026

(54) APPARATUS FOR CONTINUOUS HEALTH MONITORING AND ADAPTIVE MEDICAL-RISK PREDICTION

(71) Applicant: Nishant Narayanan, Palatine, IL (US)

(72) Inventor: Nishant Narayanan, Palatine, IL (US)

(21) Appl. No.: 19/661,740

(22) Filed: Apr. 29, 2026

Related U.S. Application Data
(63) Continuation-in-part of application No. 19/171,204, filed on Apr. 5, 2025, now Pat. No. 12,651,670.

(51) Int. Cl. G16H 50/20; A61B 5/00; G16H 10/60; G16H 70/20

(52) U.S. Cl. CPC G16H 50/20; A61B 5/6824; A61B 5/7267; A61B 5/7275; G16H 10/60; G16H 70/20

(57) ABSTRACT

An apparatus and method for continuously monitoring vital signs, securely storing the collected data, and providing real-time notifications to users upon detecting abnormal vital signs. The apparatus comprises a wearable device, a sensing device integrated with the wearable device, a wristband, a server, and a cable. Additionally, the apparatus utilizes artificial intelligence (AI) and machine learning models to analyze health data collected from multiple data sources like Electronic Medical Records (EMR) systems, other personal devices, payers to accurately predict risk to critical illness and recommend preventive care options. The apparatus also provides a conversational type personal assistant to help patients manage their health risks and deliver proactive, personalized health insights to users, thereby enhancing remote patient management, quality of care, improving patient outcomes and overall healthcare efficiency.

### FIELD OF INVENTION

The present disclosure relates generally to body vital sign monitoring systems, and more particularly to an apparatus and method for continuous vital-sign monitoring, secure medical data management, AI-driven risk prediction, and personalized preventive healthcare assistance.

### BACKGROUND

Accurate and continuous measurement of vital signs is a cornerstone of effective healthcare, as deviations in parameters such as body temperature, heart rate, blood pressure, blood glucose, respiratory rate, and blood oxygen saturation often serve as early indicators of medical deterioration. Timely detection of these physiological changes enables prompt medical intervention, which is particularly crucial for individuals with chronic diseases, elderly populations, post-operative patients, and those at risk of sudden acute events such as cardiac arrhythmia, respiratory distress, or hypoglycemic episodes.

While numerous devices exist for monitoring individual vital signs including thermometers, pulse oximeters, glucometers, and blood-pressure cuffs, these devices typically operate in isolation and require manual checks or periodic measurements. This fragmented ecosystem results in scattered, incomplete, and unsynchronized health data. Existing solutions do not integrate the patient, healthcare providers, and family members into a unified monitoring framework, nor do they generate real-time alerts to caregivers when physiological parameters fall outside safe ranges. Moreover, patients often visit multiple providers, use multiple personal monitoring devices, and maintain disparate data across unconnected applications. There is no existing platform capable of combining data from wearable devices, provider systems, and payer databases to generate reliable, context-aware, and clinically actionable predictions. This lack of a holistic, longitudinal view of patient data leads to diagnostic blind spots, delayed interventions, inconsistent care coordination, and preventable adverse health outcomes, ultimately contributing to increased healthcare costs.

Accordingly, there is a growing need for an integrated, end-to-end health monitoring system capable of continuously tracking a broad spectrum of vital signs while consolidating data from heterogeneous sources including wearable devices, personal fitness trackers, electronic medical record (EMR) systems, and payer datasets. Such a system should securely store and process aggregated health information, share relevant insights with authorized clinicians, caregivers, and family members, and support early detection of abnormalities through automated alerts and context-aware thresholds. The ability to remotely access and respond to these insights would greatly enhance proactive care management and prevent medical emergencies through timely intervention.

Therefore, there is a need for a system that seamlessly monitor vital signs, glucose levels, and blood pressure of a user in real-time while securely storing and managing user-related data to ensure privacy and reliability. There is also a need for a system that send notifications to the users in response to abnormal readings or potential health risks, enabling timely intervention. Further, there is also a need for a system that continuously analyzes data collected from wearable devices, third party personal or fitness devices used by patients, electronic medical records systems, and payer data to predict risk to critical illness, and recommends preventive care options, and provide proactive, personalized health insights to users by leveraging artificial intelligence (AI) and machine learning models.

### SUMMARY OF THE INVENTION

The following presents a simplified summary of one or more embodiments of the present disclosure to provide a basic understanding of such embodiments. This summary is not an extensive overview of all contemplated embodiments and is intended to identify neither key nor critical elements of all embodiments, nor delineate the scope of any or all embodiments.

The present disclosure, in one or more embodiments, relates to an apparatus for monitoring, tracking, and storing vital signs. The apparatus comprises a wearable device, a sensing device integrated with the wearable device, a wristband, and a server.

In one embodiment, the wearable device is configured to be worn on at least one finger of a user's hand. The sensing device is configured to collect physiological data of a user at regular intervals. The wristband is configured to allow the user to wear on a wrist. The wristband comprises a processor and a non-transitory memory for storing program instructions that are executable by the processor. The processor is in communication with the sensing device.

In one embodiment, the server in communication with the processor through a network. The server is configured to receive the physiological data of the user from the wristband in real-time, and obtain user-related medical information from plurality of external data sources. The user-related medical information comprises medical-record, health-plan data, and unstructured or semi-structured medical-record files. The plurality of external data sources includes, but are not limited to, an electronic-medical-record system, a personal device, and a payer database.

In one embodiment, the server is configured to preprocess the user-related medical information to standardize formats into structured clinical features data, wherein the structured clinical features data is stored in association with the physiological data as a user state record.

In one embodiment, the server is configured to generate time-dependent structured representations of the physiological data using a data-processing unit. The time-dependent structured representations comprise tagging abrupt physiological deviations with event markers including timestamps, change-magnitude, and activity context.

In one embodiment, the server is configured to execute an artificial-intelligence module. The artificial-intelligence module is configured to process the time-dependent structured representations, the user state record, and historical data. The artificial-intelligence module comprises a domain-specific large-language model (LLM) and a machine-learning classifier. The operations of the artificial-intelligence module improve the functioning of the apparatus by reducing inference latency via cache-augmented generation, decreasing compute-cycle consumption via parameter-efficient fine-tuning, lowering bandwidth usage by transmitting summary-level insights instead of raw data, and reducing false alerts through adaptive threshold recalibration based on real-time physiological trends.

In one embodiment herein, the artificial-intelligence module executed on the server processor is configured to process the structured representations, the user state record, and historical data using the LLM and the machine-learning classifier. The artificial-intelligence module further comprises training components configured to update model parameters using structured physiological data, ontology-normalized diagnostic-code features, and historical user state records through supervised learning, reinforcement-learning policy updates, and parameter-efficient fine-tuning to improve predictive performance over time.

In one embodiment herein, the artificial-intelligence module operates the LLM using a cache-augmented generation model configured to merge pre-trained medical data with real-time data from the plurality of external data sources. A continuous-learning model is configured to perform incremental model tuning, which includes at least one of low-rank adaptation, and parameter-efficient fine-tuning, without retraining the full model. A self-attention model is configured to prioritize clinically significant physiological measurements and diagnostic-code features. Further, multimodal embedding alignment enabling correlation of physiological-signal vectors with textual medical-record tokens.

In one embodiment herein, the machine-learning classifier comprises a k-nearest-neighbors (KNN) model configured to compute similarity measures between newly received feature vectors and stored exemplars to produce a discrete health-state classification.

In one embodiment, the server is configured to apply diagnostic-code prioritization by assigning higher feature weights to clinically significant diagnostic codes based on a medical-ontology mapping.

In one embodiment, the server is configured to compute a numerical risk score for at least one critical illness using outputs of the LLM and the machine-learning classifier.

In one embodiment, the server is configured to determine a health risk level for the user by comparing the numerical risk score with a personalized threshold, the personalized threshold is configured to be adaptively updated using at least one of statistical-adjustment techniques including weighted moving-average functions and recency weighting, and reinforcement-learning feedback signals derived from user compliance actions and physiological responses.

In one embodiment herein, the artificial-intelligence module is configured to automatically apply a ruleset mapping the numerical risk score and diagnostic-code clusters to a preventive-care plan that comprises graded intervention levels when the numerical risk score exceeds the personalized threshold. Further, the artificial-intelligence module is configured to initiate a remediation sequence that includes scheduled notifications transmitted to the computing device or the wristband until acknowledgment or override when the numerical risk score exceeds the personalized threshold.

In one embodiment, the server is configured to transmit at least one output that includes a notification, a recommendation, and a preventive action to a computing device and/or wristband of the user.

An embodiment of the first aspect wherein a method for adaptive medical-risk prediction using the apparatus for monitoring, tracking, and storing vital signs. At first step, real-time physiological data of a user is received by the server from the sensing device. Next, the user-related medical information is received from the plurality of external data sources. Next, the received user-related medical information is standardized into standardized data and generating the user state record. Next, the time-dependent structured representations of the physiological data are generated.

Next, a user health state is classified by computing similarity metrics between a current physiological feature vector and stored exemplar feature vectors using the machine-learning classifier. Next, the classified user health state is supplied to the LLM as a structured input token that constrains subsequent inference performed by the LLM. Next, the numeric semantic-relevance scores are generated by the LLM, corresponding to individual physiological feature dimensions.

Next, the numeric semantic-relevance scores are mapped to feature-weight coefficients and applying the feature-weight coefficients to modify the similarity metrics used by the machine-learning classifier during classification. Next, the cache-augmented generation is performed to retrieve recently ingested user-specific medical information from a dynamically updated memory cache and inject the retrieved information into an inference context of the LLM. Next, model parameters are incrementally tuned using low-rank adaptation or parameter-efficient fine-tuning.

Next, the numerical risk score for at least one critical illness is computed using outputs of the LLM and the machine-learning classifier. Next, personalized thresholds are updated using statistical-adjustment or reinforcement-learning feedback loop. In one embodiment, the artificial-intelligence module adaptively updates one or more personalized threshold values associated with the user's physiological parameters. In one embodiment, the artificial-intelligence module employs the reinforcement-learning feedback loop to improve the accuracy of recommendations delivered to the user.

Next, preventive-care actions and a remediation sequence are triggered when the numerical risk score exceeds the personalized thresholds. Later, physiological parameters, risk scores, preventive-care options, and remediation outcomes are displayed over the computing device.

An embodiment of the first aspect wherein the feature-weight coefficients applied to modify the similarity metrics are updated at least one of periodically, per inference window, or in response to detection of a physiological anomaly or receipt of new user-related medical information.

An embodiment of the first aspect wherein the feature-weight coefficients are updated during successive classification operations such that each classification uses a most recently generated set of feature-weight coefficients.

An embodiment of the first aspect wherein the machine-learning classifier comprises a k-nearest-neighbors (KNN) model that computes the similarity metrics using weighted distances between physiological feature vectors.

An embodiment of the first aspect wherein the server is configured to provide user-authorized access to health-related outputs through a dedicated application or web-based interface executed on a computing device of an authorized care-team member, the health-related outputs comprising at least one of numerical risk scores, predictive insights, and historical physiological data.

An embodiment of the first aspect wherein the server is configured to transmit system-generated alerts or notifications concurrently to the computing device of the user and to the computing device of the authorized care-team member when the numerical risk score exceeds a personalized threshold.

An embodiment of the first aspect wherein access to the health-related outputs is role-based and configurable by the user, such that different categories of authorized care-team members are permitted to view different subsets of data or receive different alert notifications.

An embodiment of the first aspect wherein further providing, by the server and in response to user authorization, access to health-related outputs through a dedicated application or web-based interface executed on a computing device of an authorized care-team member, the health-related outputs comprising at least one of numerical risk scores, predictive insights, and historical physiological data.

An embodiment of the first aspect wherein further transmitting system-generated alerts or notifications concurrently to a computing device of the user and to a computing device of the authorized care-team member when the numerical risk score exceeds a personalized threshold.

An embodiment of the first aspect wherein further enforcing role-based access permissions configurable by the user, such that different categories of authorized care-team members are permitted to access different subsets of the health-related outputs or receive different alert notifications.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed descriptions are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1A illustrates to a block diagram of an apparatus for monitoring, tracking, and storing vital signs, in accordance with embodiments of the invention.

FIG. 1B illustrates an illustration showing a configuration of the apparatus as worn by a user, in accordance with embodiments of the invention.

FIG. 2 illustrates a flowchart of a method for adaptive medical-risk prediction using the apparatus for monitoring, tracking, and storing vital signs, in accordance with embodiments of the invention.

FIG. 3 refers to an exemplary graphical user interface (GUI) screen for configuring a physiological parameter, in accordance with embodiments of the invention.

FIG. 4 refers to an example graphical user interface (GUI) screen presented on a mobile device for displaying initialization instructions and real-time physiological parameter readings associated with the apparatus, in accordance with embodiments of the invention.

FIG. 5 refers to an exemplary graphical user interface (GUI) screen rendered on a mobile device for presenting system-generated alerts associated with the apparatus, in accordance with embodiments of the invention.

FIG. 6 refers to an exemplary notification-configuration graphical user interface (GUI) screen for setting alert-delivery preferences associated with a monitored physiological parameter, in accordance with embodiments of the invention.

FIG. 7 refers to an exemplary graphical user interface (GUI) screen for managing user invitations within a device-management platform, in accordance with embodiments of the invention.

FIG. 8 refers to an example graphical user interface (GUI) screen used for configuring event parameters associated with a monitored measurement, in accordance with embodiments of the invention.

FIG. 9 refers to an example graphical user interface (GUI) screen showing an alert message delivered to a computing device via an electronic messaging application, in accordance with embodiments of the invention.

FIG. 10 refers to illustrates an example graphical user interface presented on the computing device, in accordance with embodiments of the invention.

FIG. 11 refers to illustrates a detailed architecture and data flow of the apparatus, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description refers to the same or like parts.

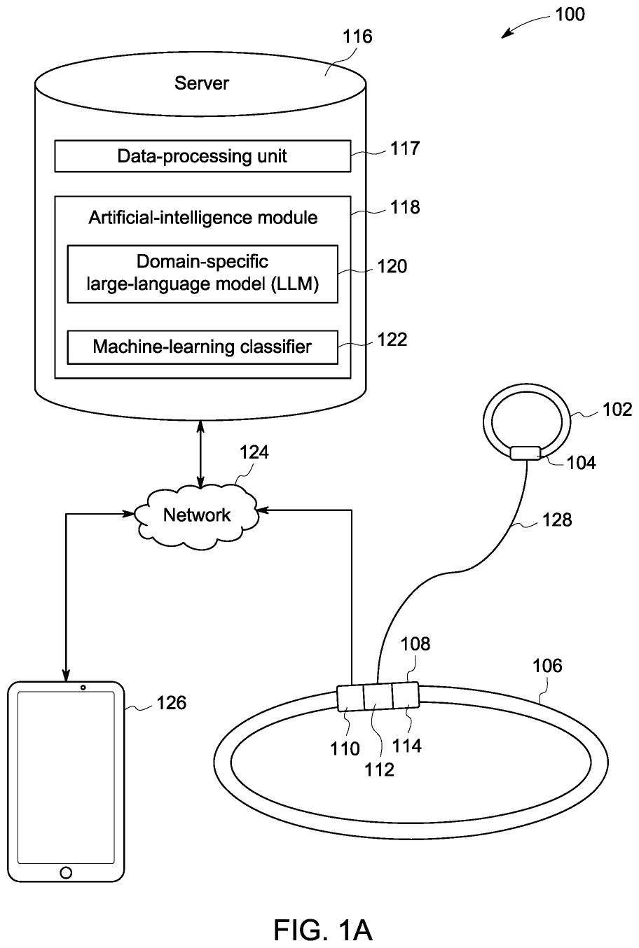

FIG. 1A refers to a block diagram of an apparatus 100 for monitoring, tracking, and storing vital signs. The apparatus 100 comprises a wearable device 102, a sensing device 104 integrated with the wearable device 102, a wristband 106, a server 116, and a cable 128. In one embodiment, the apparatus 100 includes a proprietary algorithm capable of assessing a patient's mental state resilience and hydration levels. The apparatus 100 enables health risk threshold values for vital parameters, acknowledging that normal ranges vary among individuals. Notifications are triggered based on the predefined health risk threshold values, and the AI-powered personal assistant integrates the predefined health risk threshold values for vital parameters into predictions and personalized health insights, enhancing precision in health monitoring.

In one embodiment, the apparatus 100 integrates multiple physiological parameters that include, but are not limited to, the body temperature, average oxygen level, glucose levels, blood pressure, heart rate, mental state resilience, hydration levels, and body movement, into the wearable device 102, providing a comprehensive, all-in-one health monitoring solution for patients.

FIG. 1B refers to an illustration showing a configuration of the apparatus 100 as worn by a user. In one embodiment, the wearable device 102 is configured to be worn on at least one finger of a user's hand. In a preferred embodiment herein, the wearable device 102 is implemented specifically as a ring configured to be worn on a user's finger. The ring is manufactured in a predefined range of fixed sizes and is composed of a non-conductive material such as silicone or medical-grade rubber. The ring may include a limited-size adjustment feature, such as an internal elastic insert, to accommodate minor variations in finger circumference.

In some embodiments, the wearable device 102 may be implemented in any suitable form factor, including but not limited to a ring, wristband, strap, patch, clip, or other body-worn structure configured to interface with the user's skin. The wearable device 102 may be manufactured in a wide range of sizes, shapes, or geometries and may be composed of any biocompatible, flexible, or semi-rigid material, such as silicone, rubber, polymer composites, textile fabrics, elastomers, or other conductive or non-conductive materials. The wearable device 102 may include adjustable or customizable features that enable secure placement on various body regions, such as the finger, wrist, arm, or other locations, depending on user preference and physiological-sensing requirements. In some embodiments, the apparatus 100 may include one or more ancillary wearable components, such as the wristband 106, strap, or mount, each configurable in multiple sizes and materials to support extended wearability, comfort, and accurate sensing performance.

The sensing device 104 is configured to collect physiological data of a user at regular intervals. The physiological data comprise, but are not limited to, one or more vital signs and biometrics such as heart rate, heart-rate variability, blood-oxygen saturation (SpO₂), respiratory rate, body temperature, blood-glucose level, and blood pressure. Additional physiological data may include hydration index, electrodermal activity (galvanic skin response), sleep-stage metrics derived from movement and autonomic-nervous-system signals, gait-stability measures, and activity-intensity levels. The physiological data may be sampled continuously or at predetermined intervals, and may be processed individually or in combination to characterize the user's cardiovascular status, respiratory performance, metabolic condition, autonomic balance, sleep quality, and physical-activity profile. The collected physiological data are transmitted to the server 116 for further preprocessing, feature extraction, threshold adaptation, classification, and predictive-risk modeling as described herein.

In one embodiment, the sensing device 104 comprises specific physiological sensor. The sensing device 104 outputs are mapped to derived health indicators using predefined feature associations. Mental state resilience is determined primarily from heart-rate variability features derived from cardiovascular sensing signals, optionally combined with electrodermal activity features and activity-context information derived from movement sensing. Hydration levels are determined from thermoregulatory and oxygenation-related features derived from skin-temperature measurements and blood-oxygen saturation measurements, optionally normalized using electrodermal activity and movement-derived activity intensity. These mappings enable the apparatus to compute derived health indicators from one or more physiological signals without requiring any single sensor type.

In one embodiment, the wristband 106 is configured to allow the user to wear on a wrist. The wristband 106 comprises a control unit 108. The control unit 108 comprises a processor 110 and a non-transitory memory 112 for storing program instructions that are executable by the processor 110. The processor 110 is operatively coupled to the sensing device 104 and is configured to receive, process, and interpret sensing signals generated during operation.

In one embodiment, the sensing device 104 is configured to monitor one or more physiological parameters of the user. The sensing device 104 collects the physiological data and transmits the collected data to the control unit 108. The control unit 108 forwards the physiological data to the server 116 and to a computing device 126.

In one embodiment, the computing device 126 includes a device processor and a memory storing a software module executable by the device processor. The software module may be implemented as a website or a mobile application. In some embodiments, the software module may additionally or alternatively be implemented as a plug-in component or a browser extension.

In one embodiment, the device processor communicates with the server 116 through a network 124 and is configured to execute instructions stored in the memory to enable interaction with the server 116. In certain embodiments, a database in communication with the server 116 is configured to store information related to users and user profiles. The database may include one or more program modules executable by the device processor to perform associated functions.

In some embodiments, the control unit 108 receives operating power from a connected computing device. In one embodiment, the control unit 108 includes an embedded software module that enables device management, data transmission, and communication with external systems. The control unit 108 is operably connected to the server 116 via the network 124 through a wired or wireless interface. Communication between the control unit 108 and the server 116 may be facilitated via a software application, browser extension, mobile application, web browser, operating system-integrated component, or any combination thereof installed on the computing device that interfaces with the control unit 108.

In one embodiment, the control unit 108 further comprises a power unit 114 configured to supply the required electrical power to the processor 110, the sensing device 104, and any auxiliary electronic modules. The sensing device 104 comprises one or more vitals sensors.

In one embodiment, the control unit 108 is implemented as a motherboard that integrates one or more mechanical, electrical, and electronic components for executing program logic and storing operational data. The control unit 108 may include circuit traces, microcontrollers, memory modules, signal-conditioning components, and communication interfaces for coordinating the functions of the device. The power unit 114 may include, as a non-limiting example, an on-board rechargeable battery, a removable battery cell, or a combination thereof. In certain embodiments, the power unit 114 comprises one or more solar power cells configured to harvest ambient light energy and convert it into electrical power for charging the onboard battery or directly powering the control unit 108.

In one embodiment, the cable 128 is formed from an electrically conductive material and is configured to transfer power from the power unit 114 of the control unit 108 to the sensing device 104. Exemplary conductive materials for the cable 128 include jumper wires, aluminum electrical wires, copper electrical wires, or other suitable conductive wires or cables capable of reliably delivering electrical power and data signals. The wristband 106 is fabricated from a material suitable for securely housing or supporting the control unit 108 while also providing a mechanical interface for routing and retaining the cable 128 or an associated jumper connection. Exemplary materials for the wristband 106 include silicone, rubber, textile fabrics, or other flexible non-conductive materials that ensure user comfort, durability, and electrical insulation.

In some embodiments, the cable 128 may be fabricated in various lengths and from a range of flexible or durable materials. The length of the cable 128 may be adjustable to accommodate different hand sizes and user preferences. The cable 128 is operatively connected between input and output ports of the control unit 108 and the sensing device 104, thereby enabling the control unit 108 to supply power to the sensing device 104 and support data transmission between the components. In one embodiment, the server 116 in communication with the processor 110 through the network 124. The server 116 is configured to receive the physiological data of the user from the wristband 106 in real-time, and obtain user-related medical information from plurality of external data sources. The user-related medical information comprises medical-record, health-plan data, and unstructured or semi-structured medical-record files uploaded by the user or accessed via API's. The plurality of external data sources includes, but are not limited to, an electronic-medical-record system, a personal device, and a payer database.

In one embodiment, physiological data and user-related medical information are protected using security mechanisms applied during collection, transmission, storage, and processing. Data transmitted between system components is encrypted in transit using secure communication protocols, and stored data is encrypted at rest on user devices and server-side systems. Access to data is restricted using authentication and authorization mechanisms that associate devices and users with verified credentials and access roles. Cryptographic keys are managed using key-rotation and secure-storage policies. In some embodiments, firmware and software updates are cryptographically signed and verified prior to installation to prevent execution of unauthorized code.

In one embodiment, the server 116 is configured to preprocess the user-related medical information to standardize formats into structured clinical features data. The structured clinical features data is stored in association with the physiological data as a user state record.

In one embodiment, the server 116 is configured to preprocess user-related medical information to convert heterogeneous data formats into a unified, machine-readable structure. The preprocessing pipeline may include operations such as data parsing, normalization of diagnostic codes, extraction of clinically relevant attributes, temporal alignment of medical events, and mapping of unstructured text into standardized clinical-feature vectors. The resulting structured clinical-features data is stored in association with the user's physiological data to form a user state record.

In one exemplary embodiment, when the user wears the apparatus 100, the apparatus 100 measures heart rate, blood-oxygen saturation, sleep data, and activity levels throughout the day. Further, the user-related medical information along with the physiological data is transmitted to the server 116. The user may provide the user-related medical information from electronic medical records, insurance-claim data, third-party fitness-device data, and unstructured physician notes, each of which may arrive in different formats such as PDF files, text narratives, JSON objects, API's or standardized diagnostic codes.

The server 116 receives these diverse data inputs and executes a preprocessing pipeline that performs format normalization, diagnostic-code mapping, natural-language extraction, metadata tagging, and feature-vector generation. The resulting structured clinical-feature data is stored in association with the user's real-time physiological data as a unified user state record.

In one embodiment, the user state record is implemented as a time-indexed data structure that associates physiological feature vectors, ontology-normalized diagnostic-code features, contextual metadata, and previously computed risk scores under a shared timestamp schema. The data structure supports indexed retrieval by temporal window, feature category, or diagnostic-code cluster, thereby enabling deterministic multimodal fusion and similarity computation during inference.

By way of example, consider a user diagnosed with mild asthma who uploads a hospital discharge summary in PDF format while the system concurrently receives insurance-claim data containing ICD-10 code J45.20, as well as weekly activity metrics from a connected fitness application. The server 116 extracts clinically relevant elements from the discharge summary, converts narrative information such as “recent asthma flare-up” into machine-interpretable features, aligns the extracted data with standardized diagnostic codes, and incorporates exercise-intensity metrics received from the fitness application. These combined data inputs are automatically transformed into a structured vector describing respiratory condition, medication usage, activity limitations, and correlation with sensor-derived parameters such as oxygen saturation or night-time breathing irregularities. The resulting structured clinical-features data is stored as part of the user state record, enabling an artificial-intelligence module 118 to integrate structured medical features with real-time sensor data for enhanced decision making. Using the asthma example, the artificial-intelligence module 118 may detect that the user's oxygen levels are trending lower during sleep on days with high environmental pollution and may generate a personalized alert recommending use of prescribed inhalation therapy or environmental precautions. Such adaptive insights are made possible because the server 116 first converts heterogeneous, unstructured medical information into a unified machine-readable representation that supports accurate, context-aware health inference.

In one embodiment, mappings between physiological features and derived health indicators are supported using labeled training data. Training data for mental state resilience includes physiological recordings collected during stress and recovery conditions, user-reported or clinician-annotated stress indicators, and sleep or autonomic markers correlated with heart-rate variability. Training data for hydration levels includes physiological data collected during controlled hydration and dehydration conditions, longitudinal wearable data correlated with environmental and activity context, and hydration-related ground-truth indicators such as clinician-labeled dehydration events or physiological proxies. These datasets are used to train and validate models that map physiological features to derived health indicators.

In one embodiment, the server 116 is configured to generate time-dependent structured representations of the physiological data using a data-processing unit 117. The data-processing unit 117 receives continuous or intermittently sampled physiological measurements such as heart rate, heart-rate variability, blood-oxygen saturation, respiratory rate, skin temperature, or glucose trends and transforms them into temporally aligned feature vectors. These feature vectors may include statistical summaries (e.g., moving averages, variance, short-term versus long-term trends), event markers (e.g., abrupt drops in SpO₂ or spikes in heart rate), and contextual annotations linked to activity level, sleep stage, or environmental conditions. The time-dependent structured representations produced by the data-processing unit 117 enable the artificial-intelligence module 118 to perform dynamic inference, anomaly detection, and personalized risk-score computation using consistent, clinically meaningful temporal features.

In one exemplary embodiment, when the user wears the apparatus 100 during daily activities and while sleeping. Throughout the day, the sensing device 104 collects physiological signals such as heart rate, heart-rate variability, oxygen saturation, sleep-stage transitions, and physical-activity intensity. The data-processing unit 117 receives these continuous streams of measurements and converts them into time-dependent structured representations. For instance, during a morning workout, the user's heart rate may rise sharply while oxygen saturation remains stable. The data-processing unit 117 represents this information as short-interval feature vectors that capture the magnitude of change, the rate of change, and the user's activity context. During the night, the unit may detect recurring drops in oxygen saturation aligned with transitions into deep-sleep phases, generating long-interval trend vectors and marking each event with a timestamp. These structured, time-aligned representations enable the server 116 and the artificial-intelligence module 118 to analyze physiological patterns over minutes, hours, or days, ultimately allowing detection of abnormalities such as exercise-induced stress responses or early signs of sleep-related breathing disorders.

In one embodiment, the server 116 is configured to execute the artificial-intelligence module 118. The artificial-intelligence module 118 is configured to process the time-dependent structured representations, the user state record, and historical data. The artificial-intelligence module 118 comprises a domain-specific large-language model (LLM) 120 and a machine-learning classifier 122.

In one embodiment, the classified user health state generated by the machine-learning classifier 122 is encoded as a structured input token comprising a predefined categorical identifier and an associated confidence value. The structured input token is appended to an inference context sequence provided to the LLM 120, such that the token occupies a reserved embedding position within the LLM input representation. During subsequent decoding, the structured input token influences attention weights applied to physiological feature embeddings and clinical text embeddings, thereby constraining inference in accordance with the classified health state.

In one embodiment herein, the artificial-intelligence module 118 executed on the server 116 processor 110 is configured to process the structured representations, the user state record, and historical data using the LLM 120 and the machine-learning classifier 122.

In another embodiment, the artificial-intelligence module 118 implements a technical synergy between the LLM 120 and the machine-learning classifier 122 through a bidirectional feedback loop. The machine-learning classifier 122 produces a discrete health-state classification (e.g., ‘stable’, ‘elevated risk’, ‘critical’). This classification is supplied to the LLM 120 as a structured input token, which constrains the LLM's generative process to focus on medical concepts relevant to that classified state. Concurrently, the LLM 120, through its self-attention mechanism, generates semantic-relevance scores for features in the user's medical record. These scores are fed back, due to the bidirectional feedback loop, to the machine-learning classifier 122 to dynamically adjust the feature weighting in its similarity metric calculation. This continuous, bidirectional data exchange enables a more context-aware and clinically grounded risk assessment than could be achieved by either model operating independently.

In one embodiment, the large-language model outputs semantic-relevance scores Sᵢ for individual physiological feature dimensions, where each score is normalized to a bounded interval Sᵢ∈[0,1]. Each semantic-relevance score is mapped to a corresponding classifier feature-weight coefficient wᵢ using a bounded scaling function, for example:

$${w}_{i} = {w}_{min} + ( {w}_{max} - {w}_{min} ) \cdot {S}_{i}$$

where wₘᵢₙ and wₘₐₓ are configurable parameters. This mapping increases the influence of features deemed relevant by the LLM while preventing unbounded amplification of any single feature.

In one embodiment, the feature-weight coefficients wᵢ are applied directly within a weighted similarity metric computed by the machine-learning classifier 122. For example, a weighted Euclidean distance between an input feature vector F and a stored exemplar vector E is computed as D=√(Σᵢ wᵢ (Fᵢ−Eᵢ)²). Modification of the feature-weight coefficients alters the underlying distance computation used for nearest-neighbor selection, thereby modifying classifier behavior at the machine-computation level rather than merely altering presentation of outputs.

In one embodiment, feature-weight coefficients are provided to the machine-learning classifier as a weight vector aligned with a predefined physiological feature schema. The weight vector may include associated metadata comprising a version identifier, timestamp, and applicability scope. Weight updates may occur on an event-driven basis, a periodic basis, or a combination thereof. In some embodiments, weight updates are smoothed using a recency-weighted update function to reduce oscillations and maintain classification stability during continuous monitoring.

In one embodiment herein, the artificial-intelligence module 118 operates the LLM 120 using a cache-augmented generation model configured to merge pre-trained medical data with real-time data from the plurality of external data sources. The artificial-intelligence module 118 further implements a continuous-learning model is configured to perform incremental model tuning, which includes at least one of low-rank adaptation, and parameter-efficient fine-tuning, thereby allowing targeted adjustment of selected model layers without requiring full retraining model of the LLM 120. The LLM 120 additionally a self-attention model is configured to prioritize clinically significant physiological measurements and diagnostic-code features. In some embodiments, the LLM 120 includes multimodal embedding-alignment layers that project physiological-signal vectors and textual medical-record tokens into a shared latent space, enabling accurate correlation of numerical sensor data with unstructured clinical narratives to generate context-aware insights, anomaly detections, and personalized health-risk assessments.

In one embodiment, multimodal AI inference is implemented by encoding physiological feature vectors and clinical information into numerical embeddings that are projected into a shared latent representation space. Multimodal alignment is achieved by applying projection layers that map physiological embeddings and textual or structured clinical embeddings to a common dimensionality, enabling correlation between numerical physiological patterns and medical concepts. Attention mechanisms compute relevance weights across aligned embeddings to identify physiologically and clinically significant features. Domain-specific pretraining includes training tasks that associate physiological events with clinical narratives and diagnostic-code descriptions to enable clinically meaningful interpretation during inference.

In one embodiment, multimodal alignment is reinforced during supervised or parameter-efficient fine-tuning by presenting paired training inputs comprising structured physiological feature vectors and corresponding clinical text segments or diagnostic-code descriptions. The physiological feature vectors are projected into a shared latent dimensionality with textual token embeddings, and a joint optimization objective including a language-modeling loss and a cross-modal similarity loss is applied to reduce embedding distance between physiologically and textually correlated representations. During fine-tuning, adapter or low-rank parameters of the LLM 120 may be updated while base model parameters remain fixed.

In an exemplary embodiment, the LLM 120 uniquely configured for medical analysis through interpretation and processing of multi-modal health data. The LLM 120 continuously ingests, process, and reason over streaming training data inputs. This includes real-time data from wearable devices, API's, structured and unstructured files comprising EMRs, familial medical history, and clinical observations from the care team. The data ingestion pipeline captures and delivers the data as it is generated, ensuring the model has access to the most current and contextually relevant information.

In one exemplary embodiment, when a user who recently experienced episodes of dizziness and uploads a discharge summary from an urgent-care visit while the apparatus 100 continues to stream heart-rate variability, oxygen saturation, and step-count data. The artificial-intelligence module 118 retrieves the newly uploaded clinical document and extracts diagnostic indicators such as “possible arrhythmia” and ICD-10 code 149.9. Using the cache-augmented generation model, the LLM 120 immediately integrates this fresh information with its pre-trained medical knowledge and the user's live physiological measurements. When the user begins a light evening walk, the apparatus 100 reports transient drops in heart-rate variability. The self-attention model of the LLM 120 assigns a higher weighting to these fluctuations because they correlate with the newly identified arrhythmia-related diagnostic code. The continuous-learning model performs a small incremental update, for example, low-rank adaptation to adjust internal thresholds associated with cardiac-risk classification without retraining the full model. Simultaneously, the multimodal embedding-alignment layer maps the numerical heart-rate features and textual EMR phrases into a shared latent space, enabling the LLM 120 to recognize that the observed physiological pattern may represent an early sign of exercise-induced cardiac stress. As a result, the server 116 generates an immediate risk assessment and issues a context-aware preventive recommendation advising the user to rest and consult a physician if symptoms persist.

In one embodiment herein, the cooperative operation of the artificial-intelligence module 118, the LLM 120, the data-processing unit 117, and the continuous-learning model provides specific, concrete improvements to the functioning of the apparatus 100, rather than merely performing mental steps or automating human judgment. The cache-augmented generation model reduces computational latency by enabling the LLM 120 to retrieve and incorporate recently ingested medical facts without reloading or retraining the core model, thereby improving memory efficiency and inference throughput. The parameter-efficient tuning mechanisms such as low-rank adaptation and adapter-based updates reduce processor load and eliminate the need for full model retraining, allowing the apparatus 100 to maintain real-time responsiveness even under constrained compute environments typical of wearable-device ecosystems. The self-attention model and multimodal-embedding alignment modules improve machine-level pattern recognition by enabling the apparatus 100 to correlate heterogeneous inputs numerical time-series signals and textual medical records within a unified latent representation, thereby enhancing anomaly-detection accuracy beyond what conventional rule-based or manually tuned systems can achieve.

In one embodiment herein, the machine-learning classifier 122 comprises a k-nearest-neighbors (KNN) model configured to compute similarity measures between newly received feature vectors and stored exemplars to produce a discrete health-state classification. The KNN model receives time-dependent physiological feature vectors generated by the data-processing unit 117 and compares them with previously labeled training exemplars representing clinically recognized health states. Based on the computed similarity measures such as Euclidean distance, cosine similarity, or weighted distance metrics the machine-learning classifier 122 identifies a set of nearest neighbors and generates a discrete health-state classification and an associated confidence score. This classification is subsequently provided to the artificial-intelligence module for downstream fusion with contextual medical-record features, enabling real-time assessment of the user's health condition.

In one exemplary embodiment, when a user finishes a 20-minute brisk walk while wearing the apparatus 100. The apparatus 100 streams the physiological data such as heart rate, heart-rate variability, gait stability, and oxygen saturation to the data-processing unit, which converts the raw measurements into a structured feature vector representing the user's current physiological state. When this feature vector is received, the machine-learning classifier 122 compares it against stored exemplars representing various clinically recognized states such as “normal exercise response,” “elevated cardiac stress,” or “early arrhythmia pattern.” Suppose the newly generated vector exhibits an unusual combination of decreased heart-rate variability, mild gait instability, and delayed recovery of oxygen saturation. The KNN model identifies its closest neighbors as exemplars labeled “potential post-exercise arrhythmia risk.” Based on these similarity scores, the classifier outputs a discrete health-state classification indicating elevated cardiac risk. This classification is then forwarded to the LLM 120, which combines it with contextual information from the user's electronic medical record (e.g., documented history of palpitations) to generate a personalized, natural-language warning advising the user to rest and monitor symptoms. This real-time chain of detection and interpretation is made possible through the KNN's rapid similarity-based evaluation of physiological patterns.

In one embodiment, the use of the machine-learning classifier 122 provides specific, concrete improvements to the functioning of the apparatus 100. Unlike conventional wearable systems that rely on static thresholds or fixed heuristic rules, the disclosed KNN model dynamically computes similarity measures using weighted multidimensional feature vectors, enabling more precise and context-aware classification of physiological states. This approach reduces false positives and false negatives by leveraging prior exemplars rather than simple rule-matching, improving classification accuracy even when the incoming data is noisy or incomplete. Additionally, because KNN inference requires no model-wide retraining, the system significantly reduces computational load and latency, which improves performance on resource-constrained wearable and mobile devices. The machine-learning classifier's 122 integration with the continuous-learning model further enhances functionality of the apparatus 100 by allowing incremental updates to similarity metrics, weighting factors, and exemplar sets without interrupting real-time monitoring. These technical improvements enhanced pattern recognition, reduced compute cycles, lower energy consumption, minimized data-transfer requirements, and improved real-time resilience collectively constitute technological advancements over prior systems.

Although certain embodiments describe a k-nearest-neighbors classifier and a large-language model, the disclosure is not limited to these architectures. In various embodiments, the machine-learning classifier may comprise convolutional neural networks, recurrent neural networks, transformer-based models, ensemble classifiers, or other supervised or unsupervised learning models. The large-language model may comprise an autoregressive architecture, an encoder-decoder architecture, or a hybrid architecture. Cache-augmented generation may be used with any such architecture and is not limited to a particular model type or retrieval mechanism.

In one embodiment, the server 116 is configured to apply diagnostic-code prioritization by assigning higher feature weights to clinically significant diagnostic codes based on a medical-ontology mapping. The server 116 retrieves diagnostic codes from the user's electronic medical records, insurance-claim data, or physician-uploaded documents, and maps each code to a corresponding clinical category using an ontology, or a proprietary medical-knowledge graph. Diagnostic codes associated with conditions of higher clinical relevance, for example, cardiac arrhythmias, respiratory disorders, or metabolic-risk indicators are assigned elevated weighting factors relative to routine or low-priority codes. These weighted diagnostic features are incorporated directly into the feature vectors processed by the machine-learning classifier and the LLM 120, enabling the artificial-intelligence module 118 to emphasize data that is medically significant during inference, thereby improving risk-score accuracy, anomaly detection, and context-aware health predictions.

In some embodiments, the server 116 implements the medical-ontology mapping, which is configured to translate diagnostic codes, clinical terms, and physiological-feature labels into a standardized hierarchical representation. The medical-ontology mapping may utilize any domain-specific taxonomy, knowledge graph, or structured coding system that defines relationships among diseases, symptoms, biomarkers, procedures, risk factors, and physiological parameters. Examples of such relationships include parent-child hierarchies (e.g., “cardiac arrhythmia”-> “atrial fibrillation”), synonym sets (e.g., “hypoxia”=“low oxygen saturation”), and attribute-based groupings (e.g., “respiratory conditions” associated with oxygen saturation, respiratory rate, and sleep-stage irregularity).

In one embodiment, the medical-ontology mapping receives diagnostic codes or extracted medical-record phrases and maps them to canonical nodes in the ontology. Each node is associated with a clinical relevance score, priority weight, or risk category. The mapping process may include normalization of input terms, matching to ontology entries using keyword search, embedding-based similarity, or rule-based mapping, and assignment of one or more weighted features to the corresponding physiological parameters or model inputs. For example, diagnostic codes related to cardiac disorders may automatically elevate the weighting of heart-rate variability, resting heart rate, and oxygen-desaturation events, while codes associated with metabolic conditions may prioritize glucose trends and skin-temperature variations.

The resulting ontology-aligned representation enables the artificial-intelligence module 118 to incorporate clinically significant relationships into feature weighting, risk-scoring, and inference processes. By structuring heterogeneous medical data within a unified hierarchical framework, the ontology mapping improves interoperability with diverse medical datasets, enhances context-aware interpretation of numerical sensor data, and allows dynamic re-weighting of model features in accordance with clinical priorities.

In one exemplary embodiment, when a user who recently visited a cardiologist and received an electronic medical record containing the diagnostic code 148.0, indicating paroxysmal atrial fibrillation. When the record is uploaded or retrieved through an EMR integration, the server 116 identifies this diagnostic code and maps it to a high-priority cardiovascular-risk category using a medical-ontology database. As a result, the server 116, operating through the artificial-intelligence module 118, assigns an elevated weighting factor to the code 148.0 relative to routine diagnostic entries such as seasonal allergies or minor musculoskeletal conditions. Later, when the apparatus 100 transmits new physiological data, such as elevated resting heart rate, reduced heart-rate variability, or abrupt changes in oxygen saturation, the weighted diagnostic feature associated with atrial fibrillation significantly increases the influence of cardiac-related features in the user's feature vector. This causes the machine-learning classifier 122 and the LLM 120 to treat even subtle physiological deviations as clinically meaningful in light of the prioritized diagnostic code. Consequently, the artificial-intelligence module 118 may detect a possible early arrhythmia episode that would otherwise appear insignificant and generate a timely, personalized alert advising the user to rest or seek medical evaluation.

In one embodiment, the server 116 is configured to compute a numerical risk score for at least one critical illness using outputs of the LLM 120 and the machine-learning classifier 122. The machine-learning classifier 122 provides a discrete health-state label and associated confidence value derived from similarity evaluation of the user's physiological feature vectors, while the LLM 120 provides contextual inferences derived from multimodal data sources such as diagnostic-code features, electronic medical record narratives, historical trends, and user-specific baselines. The server 116 integrates these outputs through a risk-scoring algorithm that assigns weighted contributions to each model's output and generates a continuous numerical risk score representing the likelihood of a critical illness event such as cardiac arrhythmia, respiratory distress, metabolic instability, or other medically significant conditions. The computed risk score may be dynamically updated as new physiological data is received, enabling real-time monitoring and early detection of adverse health conditions.

In one exemplary embodiment, when a user with a previously documented history of borderline hypertension and occasional palpitations. Throughout the day, the apparatus 100 streams the physiological data such as heart rate, heart-rate variability, and blood-oxygen saturation. As evening approaches, the user performs a short climbing activity, during which the data-processing unit 117 generates a feature vector indicating elevated heart rate, reduced variability, and a delayed return to baseline. The machine-learning classifier 122 identifies this feature vector as most similar to stored exemplars associated with “post-exertion cardiac strain,” producing a discrete classification and a corresponding confidence measure. Simultaneously, the LLM 120 parses the user's historical EMR records, which include diagnostic codes related to hypertension and the user's recent medical-record narrative noting intermittent chest tightness. The LLM 120 correlates these textual indicators with the live physiological trend and outputs a contextual inference indicating elevated cardiovascular concern. The server 116 combines the quantitative output of the machine-learning classifier 122 and the contextual assessment of the LLM 120 through a weighted risk-scoring function, ultimately computing a numerical risk score that exceeds the user's baseline threshold. As a result, the LLM 120 automatically issues a real-time preventive alert advising the user to rest, hydrate, and seek medical evaluation if symptoms persist.

In one embodiment, the numerical risk-score computation described improves the functioning of the apparatus 100. By combining the quantitative similarity-based health-state classifications of the machine-learning classifier 122 with the multimodal contextual reasoning of the LLM 120, the server 116 performs an integrated risk-scoring operation that cannot be carried out by conventional static-threshold systems. This fusion of heterogeneous model outputs produces more accurate, real-time predictions while reducing computational overhead, since the apparatus 100 avoids performing full-model retraining and instead relies on parameter-efficient updates. The risk-scoring algorithm further enhances performance of the apparatus 100 by enabling prioritized processing of clinically relevant features through diagnostic-code weighting, thereby reducing false alerts and unnecessary data transmissions. Additionally, the architecture allows the server 116 to update risk scores continuously as new sensor data arrives, improving temporal responsiveness without requiring continuous high-bandwidth transfer of raw data. These improvements yield measurable gains in processing efficiency, memory utilization, latency reduction, and predictive accuracy, demonstrating that the disclosed risk-scoring system constitutes an improvement to computer technology.

In some embodiments, the LLM 120 is pretrained on a domain-specific corpus comprising electronic-medical-record (EMR) narratives, diagnostic-code descriptions, peer-reviewed medical literature, and device-generated physiological sensor logs. The pretraining corpus may additionally include de-identified patient-provider conversations, care-coordination notes, and health-plan documentation to enable the model to learn contextual relationships between physiological parameters, clinical observations, and preventive-care pathways. During pretraining, the LLM 120 may be optimized using masked-token prediction, next-sentence prediction, or similar language-modeling objectives that are adapted for clinical terminology and medical-domain semantics.

After the pretraining phase, the LLM 120 is fine-tuned using supervised training examples that associate physiological-sensor data patterns and textual health summaries with target outputs, including numerical risk scores, anomaly classifications, and recommended preventive actions. In some embodiments, the fine-tuning dataset includes instruction-tuning examples comprising synthetic question-answer pairs automatically generated from medical guidelines, clinical protocols, or device-specific operating rules. This instruction-tuning process enables the LLM 120 to generate coherent, clinically aligned natural-language explanations of detected anomalies, thereby enhancing the interpretability and medical relevance of the system's real-time inference outputs.

In one embodiment, the large-language model (LLM) 120 is rendered domain-specific through pretraining and adaptation tasks that explicitly associate medical-domain inputs with clinically meaningful outputs. Domain-specific pretraining includes training objectives in which the LLM predicts masked clinical terms, diagnostic-code descriptions, or care-related concepts from electronic medical record narratives and structured clinical features, thereby learning medical vocabulary and contextual relationships. In some embodiments, the LLM is further trained using multimodal tasks in which structured physiological events or feature embeddings (e.g., oxygen desaturation events, heart-rate variability changes, or sleep-stage transitions) are provided as conditioning inputs and the model is trained to generate corresponding clinical summaries, risk interpretations, or explanatory statements. Parameter-efficient fine-tuning is applied by updating adapter parameters associated with selected layers of the LLM in response to newly ingested user state records and labeled examples, while base model parameters remain fixed, thereby enabling incremental specialization for medical-risk inference without full model retraining.

In some embodiments, the LLM 120 includes an embedding layer configured to align structured physiological-signal vectors with unstructured textual tokens. For example, physiological feature vectors derived from heart-rate-variability measurements, oxygen-saturation levels, or other sensor-based parameters may be projected into a latent space that is shared with the token embeddings of the textual medical-record inputs. This shared latent-space representation enables the attention mechanism of the LLM 120 to correlate numerical physiological features with contextual clinical narratives, thereby supporting unified multimodal inference and improving the accuracy of anomaly detection and risk-prediction outputs.

In some embodiments, model training for the LLM 120 and associated machine-learning components is executed on the server 116 cluster using distributed gradient-descent optimization techniques. The resulting model parameters, token-embedding vocabularies, and associated configuration files are stored in a non-transitory memory of the server 116. In certain embodiments, these parameters are periodically synchronized with lightweight local instances of the model executed on the wearable device or the computing device 126 to enable low-latency, near-device inference while reducing network-dependence and improving overall system responsiveness.

The domain-specific training and multimodal-embedding alignment described above enable the LLM 120 to interpret numerical physiological-signal vectors within their proper clinical context, allowing the model to generate medically meaningful insights rather than generic text outputs. By correlating structured sensor data with diagnostic-code features and medical-record narratives, the LLM 120 produces context-aware explanations, anomaly interpretations, and preventive-care recommendations that align with real-world clinical reasoning. This configuration improves the operation of the health-monitoring system by reducing false alerts through more accurate contextual interpretation, enabling near-device generation of concise, actionable explanations, and decreasing both communication bandwidth and response latency by minimizing the need to transmit full sensor datasets to the server 116.

In one embodiment, the server 116 is configured to determine a health risk level for the user by comparing the numerical risk score with a personalized threshold. The personalized threshold is dynamically updated to reflect the user's evolving physiological patterns and medical context. The personalized threshold is configured to be adaptively updated using at least one of statistical-adjustment techniques and recency weighting, and reinforcement-learning feedback signals derived from user compliance actions and physiological responses. The statistical-adjustment techniques including weighted moving-average functions and recency weighting, and reinforcement-learning feedback signals derived from user compliance actions and physiological responses.

In some embodiments, the threshold is adaptively updated using the statistical-adjustment techniques such as weighted moving-average functions and recency-weighted computations that give greater influence to the most recent physiological measurements. In other embodiments, the personalized threshold is refined using reinforcement-learning feedback signals derived from user-behavior patterns, including compliance with recommended actions, lifestyle adjustments, or observable physiological responses to previous alerts. By combining these adaptive-learning mechanisms, the system maintains an individualized, continuously calibrated threshold that improves the accuracy and responsiveness of real-time health-risk assessment.

In one exemplary embodiment, when a user who has a documented history of mild hypertension and variable sleep quality. Over several weeks, the apparatus 100 continuously streams physiological data including resting heart rate, heart-rate variability, nighttime oxygen saturation, and sleep-stage duration. The numerical risk score for cardiovascular strain is computed several times a day. Initially, the personalized threshold for classifying “elevated risk” is based on the user's baseline data collected during the first week. Over time, the server 116 observes that the user's average resting heart rate during early mornings is consistently lower than the baseline threshold due to improved sleep habits. The server 116 uses a weighted moving-average function with recency weighting to gradually shift the personalized threshold downward, ensuring it reflects the most recent physiological trend. Later, when the user responds to an alert by following a recommended breathing exercise, the system detects a rapid stabilization in heart-rate variability. This positive physiological response is treated as a reinforcement-learning feedback signal, prompting the server to adjust the threshold and policy parameters to better predict similar beneficial outcomes in the future. As a result, the adaptive threshold becomes increasingly personalized and accurate, enabling timely warnings without generating unnecessary false alerts.

The adaptive threshold-determination mechanism provides specific technical improvements to the operation of the apparatus 100. Unlike traditional monitoring systems that rely on rigid, preconfigured static limits, the disclosed threshold-updating process uses statistical-adjustment models and reinforcement-learning signals to automatically recalibrate health-risk thresholds in real time. This dynamic recalibration improves the precision of risk detection by reducing false positives and false negatives that would otherwise occur when physiological baselines shift due to age, medication changes, lifestyle improvement, or environmental factors. The use of recency weighting and weighted moving-average functions reduces computational overhead by avoiding full model retraining, enabling efficient updates that maintain low latency. Reinforcement-learning feedback further enhances system performance by identifying which user behaviors lead to clinically desirable outcomes and adjusting threshold parameters accordingly, thereby optimizing machine-level prediction accuracy. These technical improvements result in more efficient processor utilization, reduced network bandwidth consumption, and increased reliability of real-time health-monitoring functions.

In one embodiment, the adaptive threshold-updating and the reinforcement-learning feedback loops continuously calibrate internal model parameters, resulting in fewer false alerts, more stable vital-sign classification, and more efficient use of communication bandwidth by transmitting only semantically relevant updates rather than raw sensor streams. These improvements collectively enhance the performance of the server 116 and the apparatus 100 by optimizing memory utilization, reducing data-transfer overhead, enabling faster model convergence, supporting real-time inferencing, and increasing the precision of automated medical-risk predictions. As a result, the apparatus 100 achieves a technological advance over conventional monitoring systems that rely on static thresholds, non-adaptive classifiers, or offline batch processing.

In one embodiment herein, the artificial-intelligence module 118 is configured to automatically apply a ruleset mapping the numerical risk score and diagnostic-code clusters to a preventive-care plan that comprises graded intervention levels when the numerical risk score exceeds the personalized threshold. The ruleset may define varying levels of action, such as low-priority lifestyle recommendations, moderate-priority monitoring instructions, or high-priority medical escalation steps, based on the severity and clinical relevance of the combined risk score and diagnostic-code pattern.

Further, when the numerical risk score exceeds the personalized threshold, the artificial-intelligence module 118 is configured to initiate a remediation sequence that includes scheduled notifications transmitted to the computing device 126 or the wristband 106 until acknowledgment or override when the numerical risk score exceeds the personalized threshold. The remediation sequence continues until the user acknowledges the notification or until the system receives an authorized override, thereby ensuring timely user awareness and adherence to recommended preventive measures.

In some embodiments, the artificial-intelligence module 118 is configured to adaptively update one or more personalized threshold values associated with the user's physiological parameters. The adaptive update may be performed through a statistical-adjustment process or a reinforcement-learning feedback loop in which deviations between predicted values generated by the artificial-intelligence module 118 and actual physiological measurements received from the sensing device are used as feedback signals. For example, when the user's observed heart-rate variability consistently remains below an existing threshold during low-activity periods, the artificial-intelligence module 118 may automatically adjust the personalized threshold downward using a weighted moving-average function. In certain embodiments, the magnitude of the threshold adjustment is determined according to a recency-weighting factor that assigns greater influence to more recent physiological data points, thereby ensuring that the personalized threshold remains aligned with evolving user-specific physiological trends.

In some embodiments, the artificial-intelligence module 118 employs the reinforcement-learning feedback loop to improve the accuracy of recommendations delivered to the user. The artificial-intelligence module 118 interprets user actions such as compliance with hydration reminders, adherence to exercise prompts, or engagement with preventive-care instructions and the user's resulting physiological responses as feedback signals. These feedback signals are converted into reward values that are used to update policy parameters of a reinforcement-learning agent operating within the artificial-intelligence module 118. Over time, the learning agent optimizes personalized threshold values and feature-weighting parameters to promote desirable health outcomes, including stabilized vital signs, improved hydration indices, and enhanced recovery patterns. In some embodiments, the reinforcement-learning feedback loop is executed asynchronously relative to a primary inference pipeline, ensuring continuous real-time monitoring without interrupting the system's ability to compute risk scores or deliver preventive-guidance outputs.

In one embodiment, reinforcement-learning feedback signals are converted into numeric reward values r derived from observed physiological stabilization or deterioration following user compliance actions. Threshold parameters or policy parameters θ are updated according to an incremental update function θₜ₊₁=θₜ+α·r, where a represents a bounded learning-rate parameter. The reinforcement-learning update loop operates asynchronously relative to a primary inference pipeline to prevent disruption of real-time monitoring operations.

In one embodiment, the server 116 is configured to transmit at least one output that includes a notification, a recommendation, and a preventive action to the computing device 126 of the user. The transmitted output may prompt the user to take an immediate action, follow a preventive-care instruction, modify daily behavior, or seek medical evaluation, thereby enabling real-time delivery of personalized health guidance.

In some embodiments, the server 116 is configured to continuously calibrate sensor-specific offsets and user-specific baseline values using accumulated historical physiological data. The calibration parameters may be dynamically adjusted in response to changes in environmental or behavioral context, such as variations in ambient temperature, transitions between sleep states, fluctuations in stress indicators, or changes in physical-activity level. By maintaining awareness of these contextual factors, the artificial-intelligence module 118 ensures that model outputs, such as feature vectors, numerical risk scores, and personalized thresholds, remain accurate and clinically meaningful across varying operating conditions, thereby eliminating the need for manual recalibration of the wearable or associated software components.

In one embodiment, the operations of the artificial-intelligence module 118 improve the functioning of the apparatus 100 by reducing inference latency via cache-augmented generation, decreasing compute-cycle consumption via parameter-efficient fine-tuning, lowering bandwidth usage by transmitting summary-level insights instead of raw data, and reducing false alerts through adaptive threshold recalibration based on real-time physiological trends.

In one embodiment, the artificial-intelligence module 118 further comprises training components configured to update model parameters using structured physiological data, ontology-normalized diagnostic-code features, and historical user state records through supervised learning, reinforcement-learning policy updates, and parameter-efficient fine-tuning to improve predictive performance over time.

In one embodiment, the artificial-intelligence module 118 includes a model-training pipeline that comprises a pretraining stage, a multimodal-alignment training stage, a supervised fine-tuning stage, a continuous-learning stage, a reinforcement-learning stage. The pretraining stage in which the large-language model processes a corpus of structured clinical codes, unstructured medical narratives, physiological-signal logs, and device telemetry to learn domain-specific embeddings. The multimodal-alignment training stage in which physiological feature vectors are projected into a shared latent space with medical-record tokens and optimized using cross-attention reconstruction loss. The supervised fine-tuning stage using labeled training datasets linking physiological patterns and medical-record context to ground-truth risk scores and recommended actions. The continuous-learning stage in which model parameters are incrementally updated using parameter-efficient fine-tuning modules based on newly ingested user state records. The reinforcement-learning stage that updates personalized thresholds and action-selection policies using reward values derived from differences between predicted and actual physiological outcomes.

In another embodiment, the transmission of notifications, recommendations, and preventive actions from the server 116 to the computing device 126 provides specific, technical improvements to the operation of the apparatus 100. Unlike conventional systems that merely relay raw sensor data to a mobile device, the disclosed architecture performs server-side multimodal analysis, adaptive thresholding, and ruleset-based intervention generation before any data is transmitted. This reduces network bandwidth usage, lowers device-side processing burdens, and minimizes latency by delivering only clinically relevant, compressed outputs rather than full sensor datasets. The server's ability to selectively generate and transmit actionable outputs also enhances system reliability by ensuring that time-critical alerts reach the user through a prioritized communication channel, regardless of fluctuations in data volume or device capability. Furthermore, the preventive-action instructions are generated through machine-learning-derived insights, producing more accurate and context-aware recommendations than static or manually configured systems.

In one embodiment, the computing device 126 may include, but is not limited to, a smartphone, laptop, desktop computer, tablet, mobile phone, or any other suitable mobile or electronic device, and may additionally encompass virtual machines or cloud-hosted computing environments. In one embodiment, the network 124 may include, without limitation, Bluetooth, Wi-Fi, a wireless local area network (WLAN), radio-frequency communication, or other wireless communication standards or protocols. In some embodiments, a software module implemented as a platform application or browser extension is installed on the computing device 126, which is operably connected to the network 124 through either a wired or wireless interface. In one embodiment, the computing device 126 is configured to access the server 116 via the network 124. In another embodiment, communication between the computing device 126 and the server 116 is facilitated through a software application, browser extension, mobile application, web browser, operating system-integrated component, or any combination thereof.

In preferred embodiment, the server 116 is configured to receive the physiological data from the sensing device 104 and receive user-related medical information from plurality of external data sources, wherein the user-related medical information comprising structured records, unstructured medical narratives, and diagnostic-code data.

The server 116 is configured to standardize the received user-related medical information into time-aligned feature representations and store the standardized data with the physiological data as the unified user state record.

The server 116 is configured to generate, by the data-processing unit 117, time-dependent structured representations of the physiological data. The time-dependent structured representations comprise temporally aligned feature vectors including statistical summaries, event markers, and contextual annotations.

The server 116 is configured to classify a user health state using the machine-learning classifier 122 configured to compute similarity measures between newly received feature vectors and stored exemplars to produce a discrete health-state classification and confidence value.

The server 116 is configured to supply the classified health state to the LLM 120 as a structured input token, and receive from the LLM 120 semantic-relevance scores that dynamically adjust weighting factors of similarity metrics used by the machine-learning classifier 122, thereby forming the bidirectional feedback loop between the machine-learning classifier 122 and the LLM 120.

The server 116 is configured to generate predictive insights using the LLM 120 by performing multimodal fusion of physiological features and textual medical-record tokens through a latent-space embedding-alignment layer configured to project both modalities into a shared representation space, cache-augmented generation (CAG) that retrieves recently ingested medical facts from a dynamically updated memory cache and merges the retrieved facts with pre-trained model parameters to reduce inference latency, and context-aware attention weighting that prioritizes clinically significant physiological measurements and diagnostic-code features.

The server 116 is configured to update personalized thresholds and internal model parameters using a continuous-learning model. The continuous-learning model comprises recency-weighted statistical-adjustment functions applied to the user's historical physiological trends, and reinforcement-learning feedback signals derived from user-compliance actions and resulting physiological responses, the feedback signals being converted into reward values used to update internal policy parameters.

The server 116 is configured to perform parameter-efficient fine-tuning of selected LLM layers using low-rank adaptation or adapter-based modules to enable real-time tuning under wearable-device compute constraints without full model retraining.

The server 116 is configured to determine whether a predictive output exceeds the personalized threshold and, when the condition is satisfied, generate a preventive-care plan mapped from the risk score and diagnostic-code clusters, the preventive-care plan comprising graded intervention levels.

The server 116 is configured to transmit, to the computing device 126 of the user, a set of outputs including a notification, a recommendation, and at least one preventive action. The selective transmission of summarized insights in place of raw sensor streams reduces bandwidth consumption and improves real-time responsiveness of the apparatus 100.

The server 116 automatically adjusts sensor offsets based on environmental or behavioral context, including ambient temperature, sleep state transitions, and stress-indication signals.

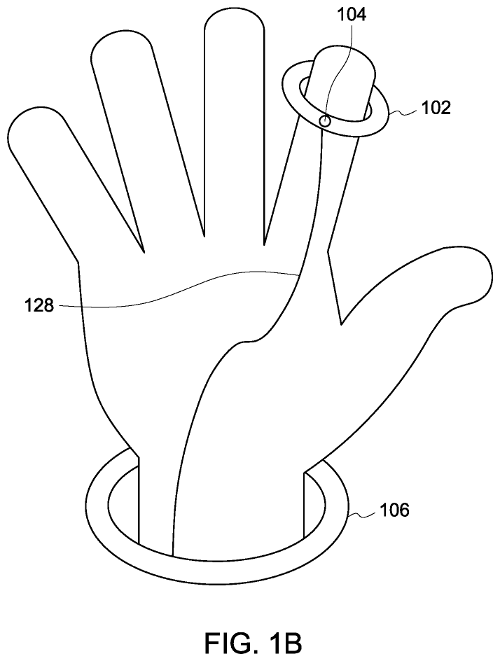

FIG. 2 refers to a flowchart 200 of a method for adaptive medical-risk prediction using the apparatus 100 for monitoring, tracking, and storing vital signs. At step 202, real-time physiological data of a user is received by the server 116 from the sensing device 104.

At step 204, the user-related medical information is received from the plurality of external data sources. At step 206, the received user-related medical information is standardized into standardized data and generating the user state record. At step 208, the time-dependent structured representations of the physiological data are generated.

At step 210, a user health state is classified by computing similarity metrics between a current physiological feature vector and stored exemplar feature vectors using the machine-learning classifier 122. At step 212, the classified user health state is supplied to the LLM 120 as a structured input token that constrains subsequent inference performed by the LLM 120. At step 214, the numeric semantic-relevance scores are generated by the LLM 120, corresponding to individual physiological feature dimensions.

At step 216, the numeric semantic-relevance scores are mapped to feature-weight coefficients and applying the feature-weight coefficients to modify the similarity metrics used by the machine-learning classifier 122 during classification.

At step 218, the cache-augmented generation is performed to retrieve recently ingested user-specific medical information from a dynamically updated memory cache and inject the retrieved information into an inference context of the LLM 120.

At step 220, model parameters are incrementally tuned using low-rank adaptation or parameter-efficient fine-tuning.

At step 222, the numerical risk score for at least one critical illness is computed using outputs of the LLM 120 and the machine-learning classifier 122. At step 224, personalized thresholds are updated using statistical-adjustment or reinforcement-learning feedback loop. In one embodiment, the artificial-intelligence module 118 adaptively updates one or more personalized threshold values associated with the user's physiological parameters. In one embodiment, the artificial-intelligence module 118 employs the reinforcement-learning feedback loop to improve the accuracy of recommendations delivered to the user.

At step 226, preventive-care actions and a remediation sequence are triggered when the numerical risk score exceeds the personalized thresholds. At step 228, physiological parameters, risk scores, preventive-care options, and remediation outcomes are displayed over the computing device 126.

In one embodiment, the feature-weight coefficients applied to modify the similarity metrics are updated at least one of periodically, per inference window, or in response to detection of a physiological anomaly or receipt of new user-related medical information.

In one embodiment, the feature-weight coefficients are updated during successive classification operations such that each classification uses a most recently generated set of feature-weight coefficients.

In one embodiment, the machine-learning classifier 122 comprises a k-nearest-neighbors (KNN) model that computes the similarity metrics using weighted distances between physiological feature vectors.

In one embodiment, the server 116 is configured to provide user-authorized access to health-related outputs through a dedicated application or web-based interface executed on a computing device of an authorized care-team member, the health-related outputs comprising at least one of numerical risk scores, predictive insights, and historical physiological data.

In one embodiment, the server 116 is configured to transmit system-generated alerts or notifications concurrently to the computing device 126 of the user and to the computing device 126 of the authorized care-team member when the numerical risk score exceeds a personalized threshold.

In one embodiment, the access to the health-related outputs is role-based and configurable by the user, such that different categories of authorized care-team members are permitted to view different subsets of data or receive different alert notifications.

In one embodiment, further providing, by the server 116 and in response to user authorization, access to health-related outputs through a dedicated application or web-based interface executed on the computing device 126 of an authorized care-team member, the health-related outputs comprising at least one of numerical risk scores, predictive insights, and historical physiological data.

In one embodiment, further transmitting system-generated alerts or notifications concurrently to the computing device 126 of the user and to the computing device 126 of the authorized care-team member when the numerical risk score exceeds a personalized threshold.

In one embodiment, further enforcing role-based access permissions configurable by the user, such that different categories of authorized care-team members are permitted to access different subsets of the health-related outputs or receive different alert notifications.

In another embodiment, the physiological data acquired from the sensing device 104 is segmented into fixed or adaptive temporal windows, each window comprising a plurality of raw sensor samples corresponding to a predefined sampling interval. Within each temporal window, the data-processing unit 117 computes statistical descriptors including at least one of a mean value, variance, slope, rate-of-change, and frequency-domain features obtained through spectral or wavelet analysis. Abrupt physiological deviations are detected by comparing the computed descriptors against baseline reference values derived from the user's historical physiological trends using thresholding, z-score analysis, or change-point detection algorithms. Upon detection of a deviation exceeding a predefined or adaptive threshold, an event marker is generated and associated with the corresponding temporal window, the event marker comprising a timestamp, a quantified change magnitude, and an activity context inferred from concurrent motion or behavioral signals.

The server 116 preprocesses user-related medical information received from external data sources by parsing structured records and applying natural-language processing pipelines to unstructured or semi-structured medical narratives to extract clinically relevant entities. Extracted entities are normalized using standardized medical ontologies including at least one of ICD, SNOMED, or LOINC to generate ontology-aligned feature vectors. The normalized clinical features are temporally aligned with the physiological feature vectors and stored in a unified user state record, the user state record representing a time-indexed data structure that associates physiological measurements, diagnostic-code features, and contextual metadata for subsequent inference and longitudinal analysis.

In another embodiment, diagnostic codes obtained from the user-related medical information are mapped to hierarchical medical ontologies, where each diagnostic code is associated with a clinical significance level based on ontology depth, category, or predefined severity annotations. The artificial-intelligence module 118 assigns feature-weight coefficients to the diagnostic-code features as a function of the associated clinical significance level, such that diagnostic codes corresponding to high-risk or chronic conditions exert greater influence on downstream classification and risk-scoring operations than lower-severity diagnostic codes.

In one embodiment, the LLM 120 generates numeric semantic-relevance scores corresponding to individual physiological and clinical feature dimensions based on contextual analysis of the user state record. The semantic-relevance scores are mapped to feature-weight coefficients that are applied to modify similarity metrics computed by the machine-learning classifier 122 during classification, such that feature dimensions deemed clinically significant by the LLM 120 contribute proportionally more to the similarity calculation. The resulting classification output is subsequently provided back to the LLM 120 as a structured input token, thereby forming a bidirectional feedback loop in which inference outputs of the LLM 120 directly influence operation of the machine-learning classifier 122.

In another embodiment, the artificial-intelligence module 118 includes a dynamically updated memory cache configured to store recently ingested user-specific medical facts, normalized clinical features, and recent inference outputs. During inference, the LLM 120 retrieves relevant entries from the memory cache based on semantic similarity or temporal proximity and injects the retrieved entries into an inference context window together with pre-trained model parameters. By reusing cached user-specific information instead of recomputing representations from raw data, the cache-augmented generation process reduces inference latency and computational overhead while preserving contextual continuity across successive inference cycles.

In another embodiment, the artificial-intelligence module 118 employs a reinforcement-learning update loop operating asynchronously with a primary inference pipeline. User-compliance actions, including acknowledgment of alerts, adherence to recommendations, or completion of preventive-care actions, are monitored and correlated with subsequent physiological responses to derive reward signals. The reward signals are converted into numeric reward values that are applied to update internal policy parameters governing threshold adjustment, alert frequency, or intervention prioritization, without interrupting real-time physiological monitoring or inference operations.

In another embodiment, to enable continuous adaptation without full model retraining, the artificial-intelligence module 118 performs parameter-efficient fine-tuning of selected layers of the LLM 120 using low-rank adaptation modules or adapter-based sub-networks. The low-rank adaptation modules introduce a limited set of trainable parameters that are updated using recent physiological feature vectors and normalized diagnostic-code features, while the remaining pre-trained parameters remain fixed. This configuration enables real-time or near-real-time model tuning under wearable-device or server compute constraints with reduced memory usage and compute-cycle consumption.

In another embodiment, personalized thresholds used to determine health risk levels are adaptively recalibrated by applying recency-weighted statistical adjustment functions to historical physiological trends in combination with reinforcement-learning feedback signals. Threshold values are increased or decreased based on observed false-positive or false-negative alert outcomes and corresponding user responses, thereby reducing alert fatigue while maintaining sensitivity to clinically significant physiological deviations.

In one embodiment, the server 116 selectively transmits summarized insights comprising classification labels, risk scores, and event markers to the computing device of the user in place of continuous raw sensor streams. By transmitting compressed, classification-level summaries rather than high-frequency physiological data, the apparatus reduces network bandwidth consumption and improves real-time responsiveness without compromising clinical relevance of the transmitted information.

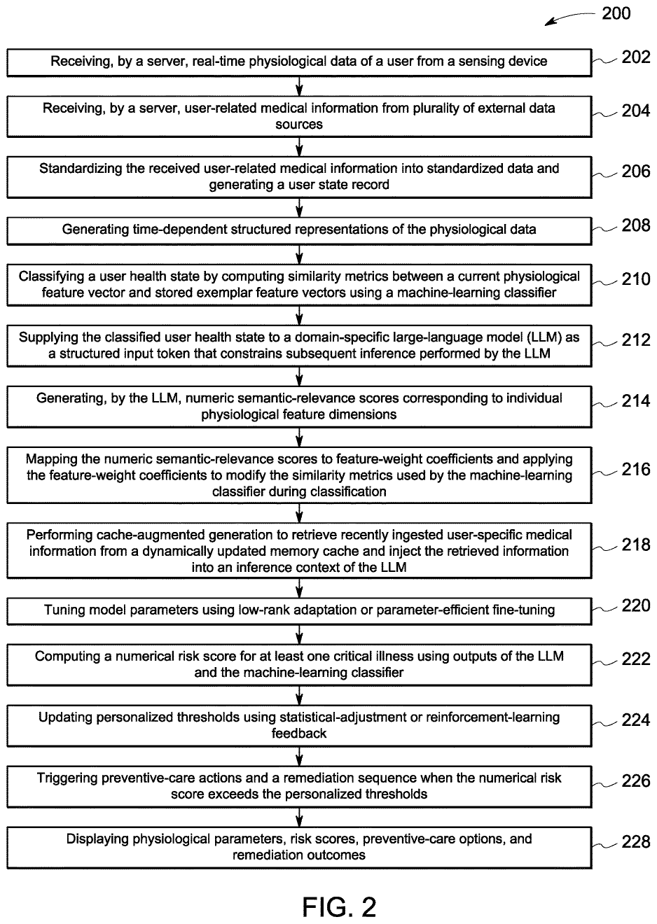

FIG. 3 refers to an exemplary graphical user interface (GUI) screen 300 for configuring a physiological parameter. The GUI screen 300 for the user of the apparatus 100, or an authorized third party such as a healthcare provider, to input customized, personal vital sign information.

The GUI screen 300 includes a name field 302 and a corresponding alias field 304, each implemented as editable text boxes for defining the identifier of the monitored parameter. Adjacent to the Alias field 304 is a selectable color indicator used for assigning a display color to the parameter.

A pin selection menu 306 is provided to assign the parameter to a specific virtual input/output channel (e.g., “VO”). In some embodiments, a unique identifier is a particular symbol used to identify the individual vital sign that the artificial-intelligence module 118 determines and cannot be customized by the user.

A data type dropdown menu 308 allows the user to specify the data format or type of data for the parameter, such as “Integer.” In some embodiments, the data format is determined by the artificial-intelligence module 118, cannot be customized by the user, and identifies the type of data the server 116 is collecting.

A units field 310 enables the user to select the measurement unit, illustrated here as “Percentage, %”. In some embodiments, the measurement unit is determined by the server 116, cannot be customized by the user, and identifies the type of unit of the data format collected by the server 116.

A min range entry region 312 and a max range entry region 314 permits definition of allowable minimum and maximum parameter values (e.g., 95 and 100, respectively). A default value selector 316 allows the user to define a pre-set default reading (e.g., 97). A toggle switch 318 labeled Enable history data allows activation of historical data logging functions. Beneath this section is an advanced settings expandable region 320 that includes additional system-level options. These options include: a Save raw data toggle with an “Upgrade” indicator, an Invalidate in field allowing the user to set an expiration interval (e.g., 5 seconds) and select a post-expiration action (e.g., “Nothing”), a Sync with latest server value every time device connects to the cloud toggle, a Show in custom charts toggle, and a Show in reports toggle.

In some embodiments, the toggle switch 318 allows the mobile dashboard and/or web platform to store historical vitals data. An example of advanced settings includes displaying “Nothing” or “No Data” when the mobile dashboard or web platform has not received data in a set amount of time (e.g., 1 minute). Another example of advanced settings includes syncing with latest server value every time device connects to the cloud. When this setting is turned on, the apparatus 100 that has been offline will retrieve the latest value collected from the sensing device 104 when the apparatus 100 turns back online. The control unit 108 will then collect data regularly. Another example of advanced settings includes showing vitals data in custom charts and reports for the user, health care team and/or doctor to see vitals data over a period of time. In some embodiments, the collected vitals data can be plotted in a graph to show the patient's progress related to that particular vital sign. In some embodiments, the collected vitals data can be used to show improvement or decline related to the particular vital sign, as well as the rate of acceleration or deceleration of the improvement or decline. In some embodiments, the collected vital signs can be used to provide an overview of a patient's vital signs mapped against the patient's circadian rhythm. Over time, if the vital signs are not following the patient's circadian trend, the system can alert the health care team of possible sleep disturbance.

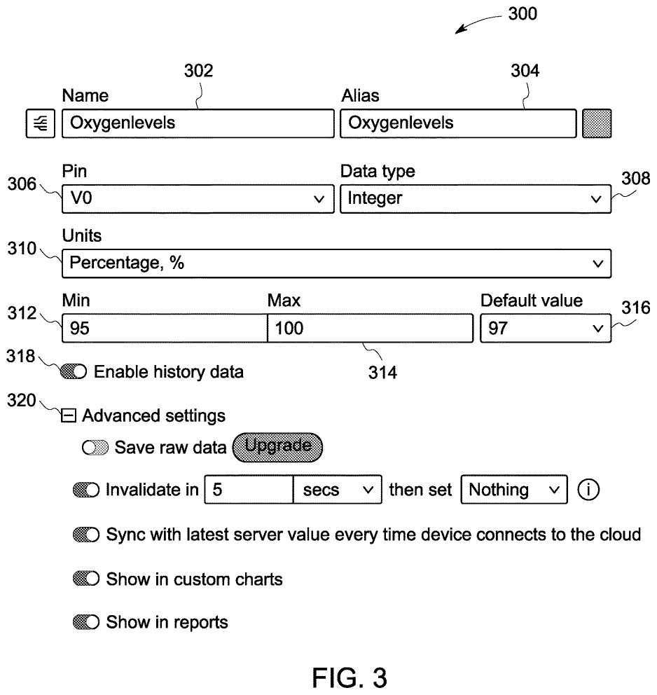

FIG. 4 refers to an example graphical user interface (GUI) screen 400 presented on a mobile device for displaying initialization instructions and real-time physiological parameter readings associated with the apparatus 100. The top portion of the GUI screen 400 includes a header section labeled Quickstart device is displayed, which includes a back-navigation icon and additional settings icons.

A message panel is positioned below the header and provides onboarding instructions. The panel includes a bold instruction “Now go to your computer.” followed by a secondary instruction prompting the user to check their inbox for a setup code and further steps to bring the apparatus 100 online. The message panel also contains an illustrative graphic representing a computer screen.

The lower portion of the GUI screen 400 displays multiple physiological measurements generated by the device. A first parameter region 402 presents Body temperature alongside a corresponding numerical value displayed as 89.83° F. Adjacent to this, a second parameter region 404 presents Oxygen level with a displayed value of 70.

Further below, a third parameter region 406 displays Mental state resilience, represented qualitatively as GOOD. A fourth parameter region 408 presents Heart rate with a displayed numerical value of 166. A fifth parameter region 410 displays Hydration levels, represented qualitatively as OK.

The data shown on the GUI screen 400 can be the last sample collected by the sensing device 104. In other embodiments, it can display the average value of collected samples over the previous minute, hour, 6-hour period, 12-hour period, or day. A display reading of “GOOD” for mental state resilience indicates minimal or no variability in heart rate. A display reading of “POOR” for mental state resilience indicates abnormal variability in heart rate. A display reading of “OK” for hydration levels indicates regular body temperature and regular oxygen levels. A display reading of “POOR” for hydration levels indicates high body temperature and low oxygen levels.

The GUI screen 400 allows a user to view onboarding instructions and simultaneously monitor multiple health-related parameters communicated by the sensing device. The various labeled regions, numerical indicators, and qualitative descriptors provide an organized, multi-metric summary of user health status on a single screen.

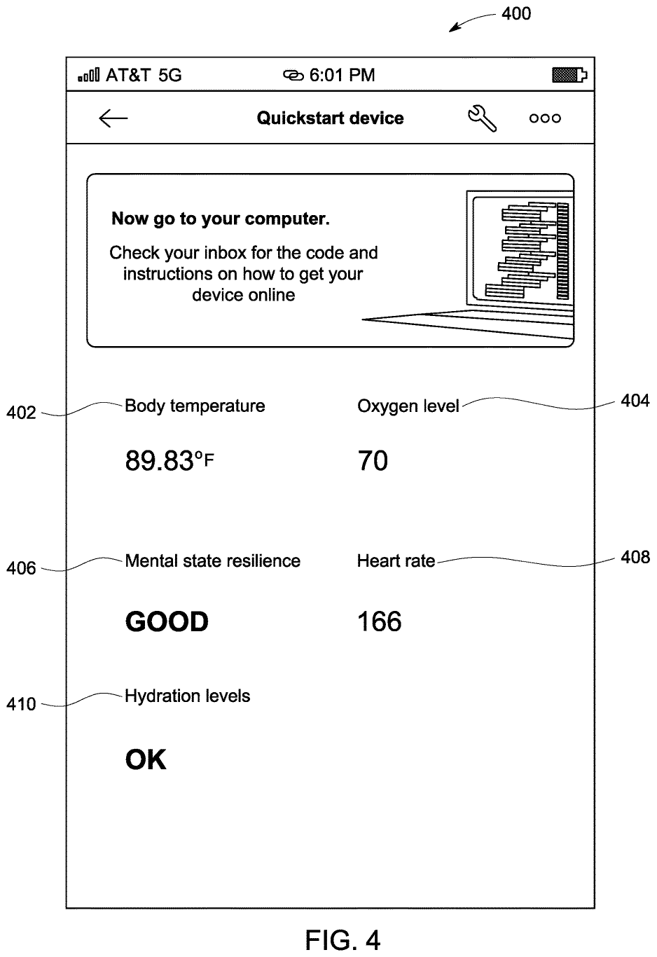

FIG. 5 refers to an exemplary graphical user interface (GUI) screen 500 rendered on a mobile device for presenting system-generated alerts associated with the apparatus 100. The top portion of the GUI screen 500 includes a header region displays a user-profile icon, the user's identifier (shown as “B”), and the application name (“Blynk”).

A section labeled Alerts is positioned below the header and contains multiple alert notifications, each corresponding to a detected physiological condition or device-generated warning. Notifications are sent to a mobile dashboard when vital signs are outside of the inputted range. Exemplary notification for low oxygen level is displayed in a first alert panel 502 includes a label identifying the originating device (quickstart device), a timestamp (“Today at 6:42 PM”), and an alert message corresponding to an oxygen level warning. The message indicates that the oxygen level is low and advises the user to use an oxygen mask if available. A second alert panel provides a similar oxygen-level warning issued earlier at 6:36 PM.

Further down, a third alert panel presents a heartbeat alert from the same device, timestamped at 6:36 PM. The associated message notifies the user of a high heart rate and includes a recommended action (“CALM DOWN!!! REST UP!!!”). An additional heart-rate alert with the same warning is displayed immediately below it, timestamped at 6:35 PM. A final alert panel at the bottom of the list shows another device notification timestamped at 6:33 PM.

At the bottom of the GUI screen 500, a navigation bar provides selectable icons enabling access to devices, automations, and notifications features within the application. In some embodiments, a notification is sent when oxygen levels drop for over 30 seconds, one minute, two minutes, five minutes, ten minutes, or longer. In some embodiments, a notification is sent when hydration levels decrease over the course of 10 minutes, 20 minutes, 30 minutes, 40 minutes, 50 minutes, 60 minutes, or longer. In some embodiments, a notification is sent immediately when heart rate variability indicates atrial fibrillation or other severe cardiac conduction abnormality.

The GUI screen 500 consolidates time-stamped physiological alerts and recommended user actions into a structured, scrollable layout, enabling real-time monitoring and rapid response to health-related conditions.

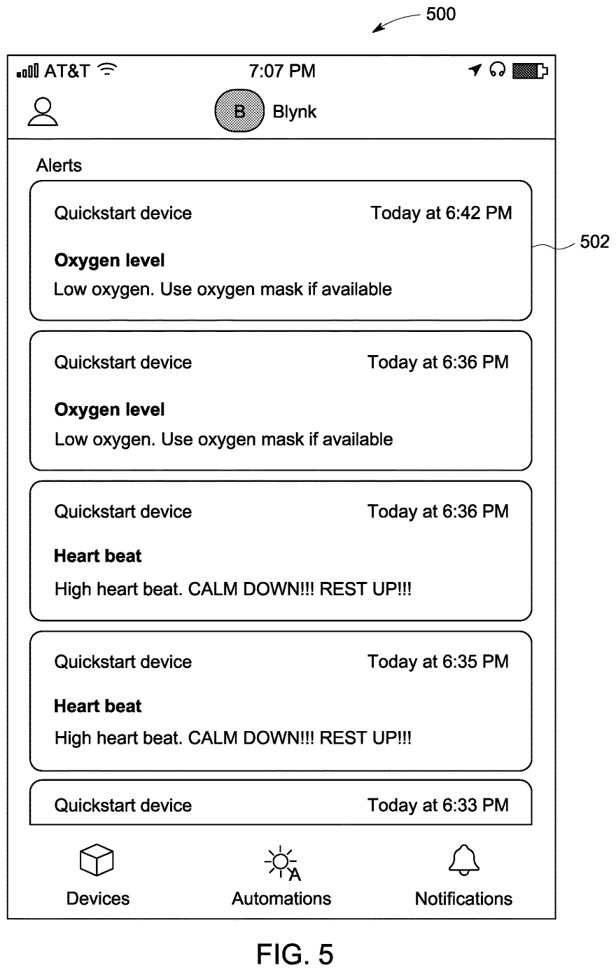

FIG. 6 refers to an exemplary notification-configuration graphical user interface (GUI) screen 600 for setting alert-delivery preferences associated with a monitored physiological parameter. The GUI screen 600 includes two selectable tabs, General and Notifications.

A toggle switch is provided at the top of the notification settings section to enable notifications. When enabled, a default recipients section becomes available, allowing the user to define one or more communication channels for receiving automated alerts. The interface includes an E-mail To field 602, implemented as a selectable contact-entry field for designating an email recipient. A corresponding push notification to field 604 allows the user to select a contact to receive push-based alerts through the associated mobile application. A further SMS To field 606 enables designation of a recipient for text-message-based alerts.

At the bottom of the GUI screen 600, an additional toggle option labeled deliver push notifications as alerts allows the user to specify whether push notifications should also be escalated into higher-priority alert messages. The GUI screen 600 thus provides a centralized configuration panel through which users can customize multi-channel alert delivery for real-time monitoring of health-related parameters.

In some embodiment, the GUI screen 600 is for the user or authorized third party to set up notification alerts when the user's vital signs are out of the minimum value 450 and maximum value 460 range for a designated period of time the user or authorized third party inputted in the display of FIG. 3. In some embodiments, the input screen for setting up notifications includes a text box to input email contact information for the user and authorized third parties to receive notifications. In some embodiments, the input screen for setting up notifications includes a text box to input mobile application push notifications for the user and authorized third parties to receive notifications. In some embodiments, the input screen for setting up notifications includes a text box to input SMS text contact information for the user and authorized third parties to receive notifications.

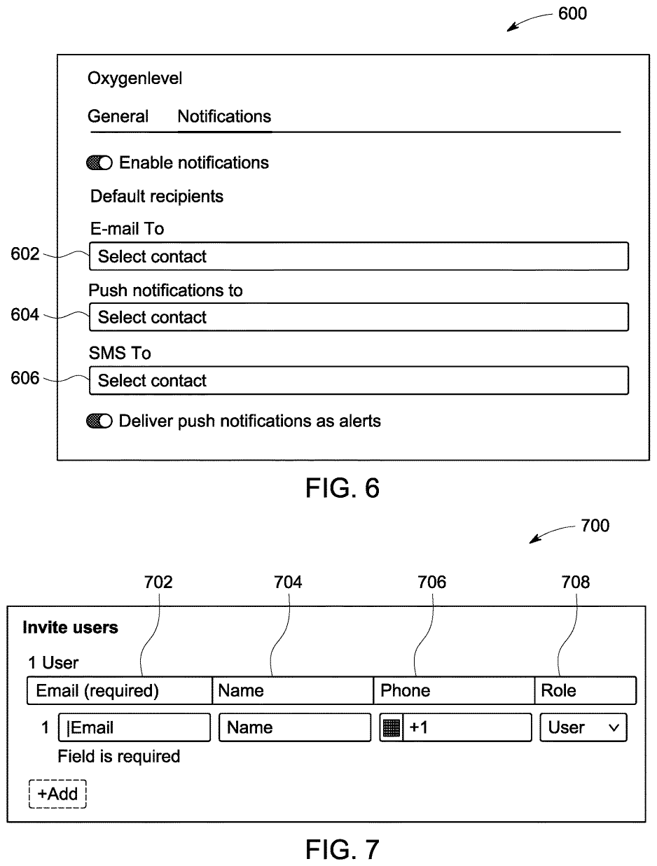

FIG. 7 refers to an exemplary graphical user interface (GUI) screen 700 for managing user invitations within a device-management platform. The GUI screen 700 includes an Invite users section configured to allow an administrator or primary user to add one or more additional users to the apparatus 100.

A header row includes multiple labeled input categories, including an email (required) field 702, a name field 704, a phone field 706, and a role field 708. In some embodiments, the role of the user is “admin” and the role of other authorized third party's is “user”.

Beneath the header row, a first user-entry row is displayed, containing interactive input fields where the email address, user name, and phone number may be entered. The Email field indicates that the field is required and displays an error prompt (“Field is required”) if left blank. The phone field includes a default country code selector (shown as “+1”). The Role field includes a dropdown menu allowing selection of predefined access roles, such as User.

A button labeled+Add is positioned below the user-entry row and is configured to allow the addition of further user rows for inviting multiple individuals. The interface allows the administrator to define and submit user information necessary for granting system access, including identification details and permission roles.

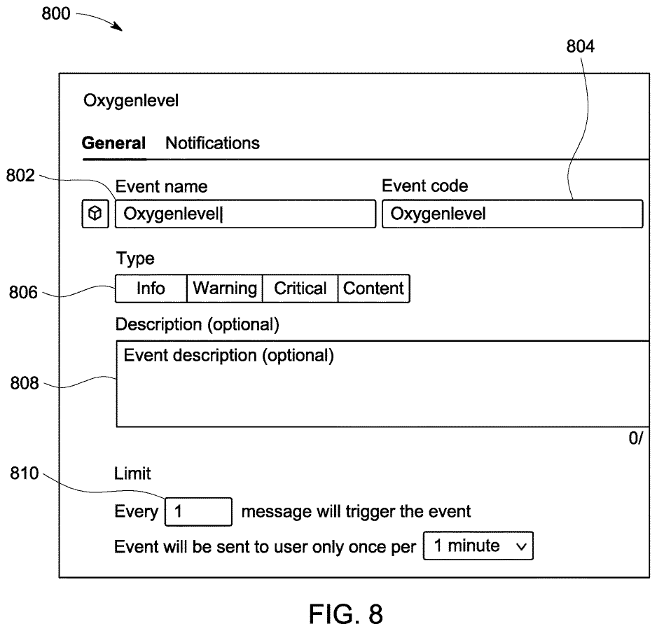

FIG. 8 refers to an example graphical user interface (GUI) screen 800 used for configuring event parameters associated with a monitored measurement. The GUI screen 800 includes two selectable tabs general and notifications.

An event-definition section includes an event name field 802 and a corresponding event code field 804, each represented as editable text-entry boxes enabling the user to assign or modify identifiers for the event. A type selection region 806 allows the user to categorize the event by choosing among multiple classification options such as info, warning, critical, or content, each presented as a selectable button.

A description region 808 provides a multi-line text field for entering a descriptive explanation of the event. A character counter (shown as “0/”) may be positioned adjacent to the description field to indicate remaining allowable characters.

A limit configuration section 810 is displayed near the bottom of the interface. This section allows the user to specify how frequently the event should be triggered. A first control permits entry of a numeric interval defining that “Every [X] message will trigger the event.” A second control specifies the minimum time interval during which the event notification will only be sent once, selectable from a dropdown menu (e.g., “1 minute”).

For example, this is where the message shown in FIG. 5, “CALM DOWN!!! REST UP!!! can be included. In some embodiments, the limit configuration section 810 is available for the user to input the limit of messages triggered to avoid excessive notifications if the associated vital sign is out-of-range for a long period of time. For example, if the user's oxygen level is low for a long period of time, the system's default setting is to send an alert or notification for each collection of samples sent from the sensing device 104 to the control unit 108. The limit will reduce the number of alerts or notifications sent to the designated amount (e.g., 1 notification per minute) even if there are multiple readings that are out of range within that designated time period.

The GUI screen 800 thereby enables detailed customization of event conditions, classifications, descriptions, and rate-limiting behaviors associated with automated alerts generated by the apparatus 100.

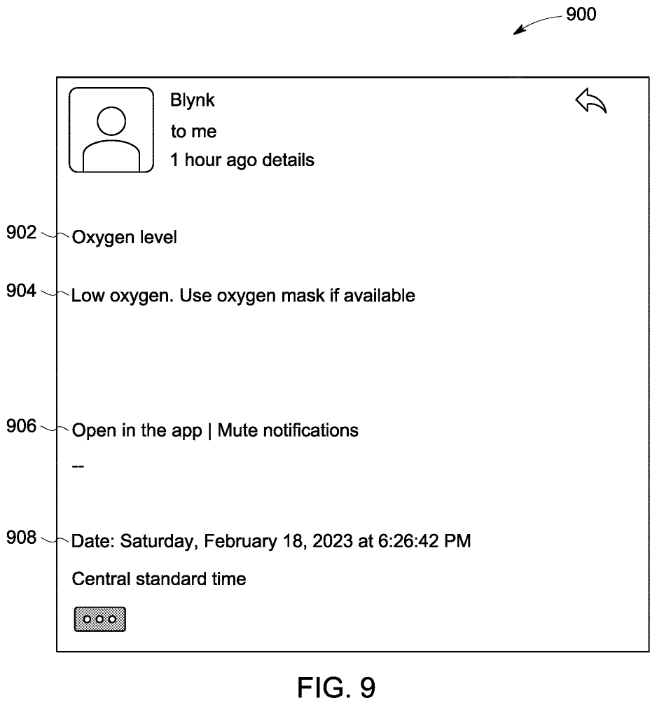

FIG. 9 refers to an example graphical user interface (GUI) screen 900 showing an alert message delivered to the computing device 126 via an electronic messaging application. The GUI) screen 900 includes a header region displaying the sender information, shown here as “Blynk,” along with an identifier indicating that the message was sent to the user and a timestamp indicating that the message was received approximately one hour earlier. A profile icon is displayed adjacent to the sender information.

A first text region 902 displays the title of the alert, shown here as oxygen level. A second text region 904 displays the alert content, which provides a corresponding advisory message, such as low oxygen. Use oxygen mask if available.

A third region 906 includes actionable links or options, such as open in the app and Mute notifications, enabling the user to access the associated application interface or modify notification settings directly from the message. A separator indicator (“--”) may be displayed below these options.

A timestamp region 908 indicates the precise date and time the alert event was generated, displayed here as Saturday, Feb. 18, 2023 at 6:26:42 PM, along with the applicable time zone (e.g., Central standard time). A button having three dots is located beneath the timestamp, which may correspond to additional message options or extended menu functions.

Overall, the GUI screen 900 demonstrates how the system delivers formatted alert messages containing sensor-specific event details, recommended actions, application shortcuts, and metadata regarding event timing.

In some embodiments, the name of the vital sign, the custom alert or message, the option to open the notification in the app or mute notifications, and the date and time of the notification will appear in the email. The email alert will depict the customized message the user entered in the input screen of FIG. 8.

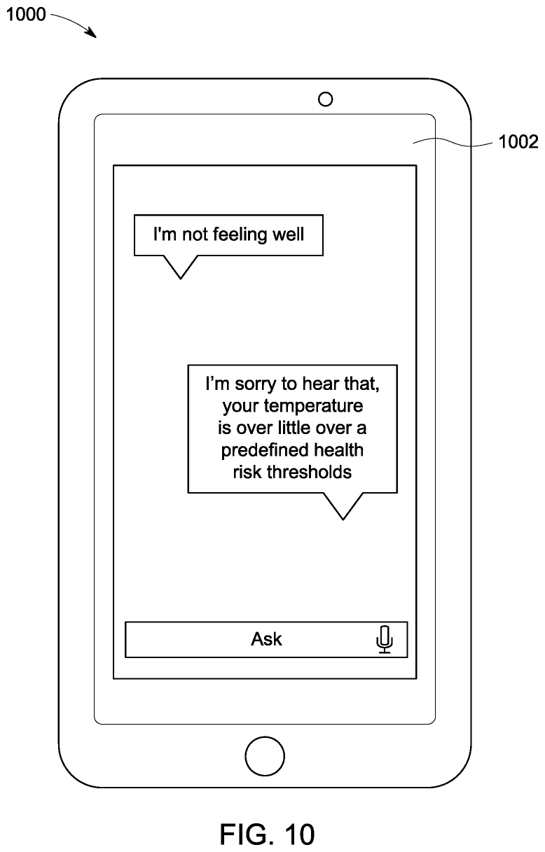

FIG. 10 refers to illustrates an example graphical user interface 1000 presented on the computing device 126, such as a smartphone or tablet. The computing device 126 includes a display screen 1002 on which a conversational interface is rendered.

The display screen 1002 presents a chat-based interaction between the user and an automated digital assistant. A first message bubble, originating from the user, displays the text “I'm not feeling well.” Below this, a second message bubble represents a system-generated response from the digital assistant. The response indicates that the user's measured health parameter exceeds a preset safety threshold, shown here as “I'm sorry to hear that, your temperature is over little over a predefined health risk thresholds.”

At the bottom portion of the interface, an input bar is displayed, allowing the user to enter additional text or queries. The input bar includes a button labeled “Ask” and a microphone icon, representing an option for voice-based input.

The graphical user interface 1000 demonstrates an example of how the system provides real-time conversational feedback to a user based on detected physiological parameters, and facilitates continued user interaction through text or voice input.

These adaptive mechanisms improve the overall functioning of the apparatus 100 by, reducing false alerts through continuous, user-specific threshold calibration performed by the artificial-intelligence module 118, minimizing retraining latency by enabling the server 116 to apply incremental, parameter-efficient model updates rather than executing full-model retraining; and extending device autonomy by permitting local or near-device inference adjustments that reduce the volume of raw data transmitted to backend servers, thereby lowering network bandwidth consumption and improving real-time responsiveness.

Additionally, the apparatus 100 utilizes artificial intelligence (AI) and machine learning models to analyze health data collected from multiple data sources like Electronic Medical Records (EMR) systems, other personal devices, payers to accurately predict risk to critical illness and recommend preventive care options. The apparatus 100 also provides a conversational type personal assistant to help patients manage their health risks and deliver proactive, personalized health insights to users, thereby enhancing remote patient management, quality of care, improving patient outcomes and overall healthcare efficiency.

In one embodiment, the computing device 126, mobile application, or web-based interface associated with the apparatus 100 is configured to enable user-authorized sharing of health-related data with one or more members of a care team. Authorized care-team members may include physicians, nurses, clinical staff, caregivers, or other designated individuals involved in the user's health management.

In some embodiments, access to shared data is provided through a dedicated application or web interface executed on the computing device 126 of the authorized care-team member. The care-team application is configured to authenticate the authorized user and present a secure, role-based view of shared health information selected by the user.

The care-team application is configured to display one or more of the user's numerical risk scores, predictive insights, historical physiological trends, and time-dependent structured representations generated by the server. In some embodiments, the care-team application presents longitudinal visualizations of physiological parameters annotated with risk-score changes, alert events, and predictive indicators to support clinical review.

In one embodiment, the care-team application is configured to receive the same or a subset of system-generated alerts, notifications, and preventive-care recommendations that are delivered to the user. For example, when the numerical risk score exceeds a personalized threshold, alert notifications may be transmitted concurrently to a user device and to one or more authorized care-team devices, thereby enabling timely awareness and coordinated response.

In some embodiments, alert delivery preferences for care-team members are configurable by the user and may include real-time alerts, periodic summaries, or event-triggered notifications associated with specific physiological parameters or risk conditions. The user may modify, suspend, or revoke alert-sharing permissions at any time.

The care-team application may further enable review of historical alert events, acknowledgment status, and associated contextual information, including contributing physiological features or trend indicators generated by the artificial-intelligence module. This functionality allows care-team members to correlate system-generated alerts with clinical observations, treatment plans, or patient-reported symptoms.

By enabling authorized care-team members to access predictive risk assessments, historical data, and shared alert notifications through a dedicated application interface, the apparatus supports collaborative care workflows and improves continuity of care. This architecture differs from conventional wearable systems that provide only passive data viewing by enabling controlled, AI-contextualized information sharing that is directly actionable by clinical personnel.

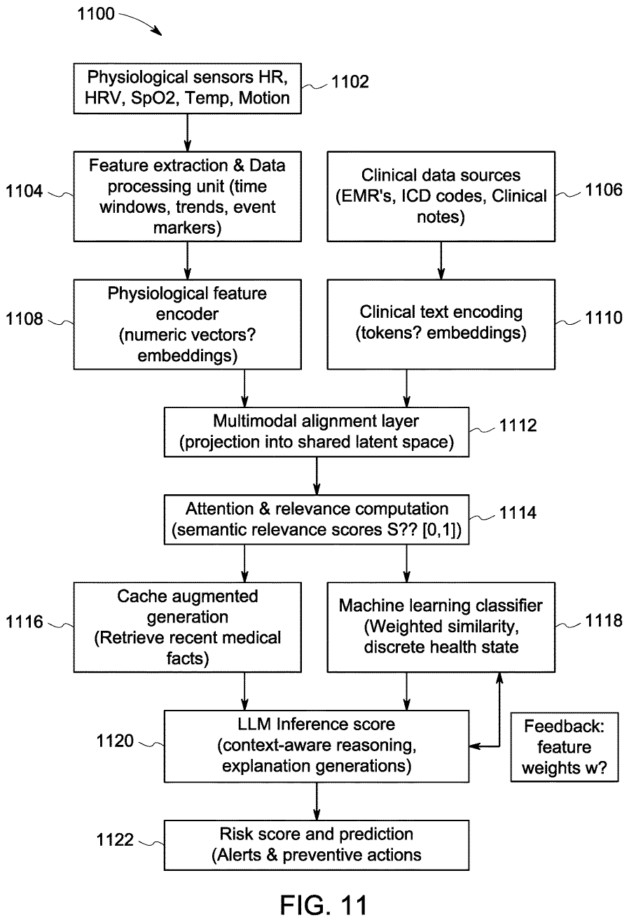

FIG. 11 refers to illustrates a detailed architecture and data flow 1100 of the apparatus 100. In one embodiment, the apparatus 100 comprises the sensing device 104. In another embodiment, the sensing device 104 comprises a plurality of physiological sensors. The sensing device 104 is configured to continuously or intermittently acquire raw physiological signals of a user. The sensing device 104 include, but are not limited to, heart rate (HR) sensors, heart rate variability (HRV) sensors, blood oxygen saturation (SpO₂) sensors, body temperature sensors, and motion or inertial sensors. At step 1102, the sensing device 104 generate time-series physiological data streams that reflect both instantaneous and longitudinal physiological states of the user. The acquired physiological signals are transmitted to the data-processing unit 117 for further analysis.

In one embodiment, the data-processing unit 117 is configured to receive the raw physiological data and generate structured physiological feature representations. The data-processing unit 117 applies predefined and adaptive processing operations including temporal windowing, trend analysis, normalization, noise filtering, and detection of physiological events. At step 1104, the extracted features may include statistical summaries, temporal gradients, variability measures, and event markers associated with physiological deviations or contextual activities. The resulting processed physiological features are structured as time-aligned numerical representations suitable for downstream encoding.

In parallel, the apparatus 100 is configured to receive user-related clinical data from one or more clinical data sources, at step 1106. The clinical data sources include electronic medical records (EMRs), diagnostic or classification codes including ICD codes, physician notes, discharge summaries, laboratory reports, and other structured or unstructured clinical documents. The clinical data may be received periodically, on-demand, or in response to detected physiological events and is stored or buffered for encoding and inference.

In one embodiment, the apparatus 100 comprises a physiological feature encoder configured to convert the processed physiological features into numerical vector representations. At step 1108, the physiological feature encoder maps the structured physiological features into fixed-length or variable-length embeddings using linear projection layers, neural encoders, or other embedding mechanisms. The resulting physiological embeddings preserve temporal and semantic relationships between physiological parameters and are optimized for multimodal correlation with clinical information.

In one embodiment, the apparatus 100 includes a clinical text encoding module configured to process the received clinical data. At step 1110, the clinical text encoding module tokenizes, embeds, and encodes unstructured and semi-structured clinical information, including clinical notes and diagnostic codes, into numerical embeddings. The encoding process may include medical-domain tokenization, ontology-based normalization, and contextual embedding using pretrained or fine-tuned language models. The resulting clinical embeddings represent the semantic content of the clinical data in a machine-interpretable form.

In one embodiment, the apparatus 100 includes a multimodal alignment layer configured to receive the physiological embeddings and the clinical embeddings and project both modalities into a shared latent representation space. At step 1112, the multimodal alignment layer applies modality-specific projection functions that map physiological feature vectors and clinical text embeddings to a common dimensionality. This shared latent space enables correlation, similarity computation, and joint reasoning across heterogeneous physiological and clinical data modalities.

In one embodiment, the apparatus 100 further includes an attention and relevance computation module operating on the aligned multimodal representations. At step 1114, the attention and relevance computation module computes semantic relevance scores corresponding to individual physiological features, clinical tokens, or aligned latent dimensions. Each relevance score is normalized within a bounded range, for example between 0 and 1, representing the relative clinical importance of the associated feature. The relevance scores are generated using attention mechanisms, similarity metrics, or learned weighting functions and are used to prioritize clinically significant inputs during inference.

In one embodiment, the apparatus 100 includes the cache-augmented generation module configured to retrieve recently ingested or user-specific medical information from a dynamically updated memory cache. At step 1116, the cache-augmented generation module retrieves relevant medical facts, recent diagnoses, or prior inference results and injects the retrieved information into the inference context. This mechanism enables the apparatus 100 to incorporate fresh, personalized medical context without reprocessing or retraining the underlying models, thereby reducing inference latency and improving contextual accuracy.

In one embodiment, the machine-learning classifier 122 is configured to compute a discrete health state of the user. At step 1118, the machine-learning classifier 122 operates on the aligned embeddings and applies weighted similarity metrics influenced by the semantic relevance scores. The machine-learning classifier 122 may comprise a k-nearest-neighbors model, distance-based classifier, or other supervised learning model that outputs a discrete health state classification and an associated confidence value. The relevance scores are mapped to feature-weight coefficients that directly modify the similarity computation performed by the machine-learning classifier 122.

In one embodiment, the LLM 120 is configured to perform context-aware reasoning over the multimodal representations. At step 1120, the LLM 120 receives inputs from the cache-augmented generation module and the machine-learning classifier 122 and generates an inference score representing contextual medical risk, anomaly interpretation, or clinical explanation. The LLM 120 generates human-readable explanations, contextual summaries, and reasoning outputs that integrate physiological patterns with clinical context.

In one embodiment, the apparatus 100 implements a bidirectional feedback mechanism between the LLM 120 and the machine-learning classifier 122. The LLM 120 outputs updated semantic relevance scores that are converted into feature-weight coefficients and fed back to the machine-learning classifier 122. This feedback dynamically adjusts the weighting of physiological and clinical features during subsequent classification operations, enabling adaptive refinement of similarity metrics and improving classification accuracy over time.

In one embodiment, the apparatus 100 computes a numerical risk score based on the outputs of the LLM 120 and the machine-learning classifier 122. At step 1122, the numerical risk score represents a predicted likelihood of a medical condition, physiological deterioration, or health anomaly. The apparatus 100 compares the risk score against one or more thresholds and generates predictive outputs including alerts, preventive recommendations, or intervention actions when the risk score exceeds a defined threshold.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. An apparatus for monitoring, tracking, and storing vital signs, comprising:
a wearable device configured to be worn on at least one finger of a user's hand;
a sensing device integrated with the wearable device, wherein the sensing device is configured to collect physiological data of a user at regular intervals;
a wristband configured to allow the user to wear on a wrist, wherein the wristband comprises a processor and a non-transitory memory storing instructions executable by the processor, wherein the processor is in communication with the sensing device; and
a server in communication with the processor through a network, wherein the server is configured to:
receive the physiological data of the user from the wristband in real-time;
obtain user-related medical information from a plurality of external data sources;
preprocess the user-related medical information to standardize formats into structured clinical features data, wherein the structured clinical features data is stored in association with the physiological data as a user state record on the server;
generate time-dependent structured representations of the physiological data using a data-processing unit, wherein the time-dependent structured representations comprise tagging abrupt physiological deviations with event markers including timestamps, change-magnitude, and activity context;
process the time-dependent structured representations, the user state record, and historical data using an artificial-intelligence (AI) module, wherein the artificial-intelligence module comprises a domain-specific large-language model (LLM) and a machine-learning (ML) classifier;
apply diagnostic-code prioritization by assigning higher feature weights to clinically significant diagnostic codes based on a medical-ontology mapping,
wherein the artificial-intelligence module comprises training components configured to: update model parameters using structured physiological data, ontology-normalized diagnostic-code features, and historical user state records through supervised learning, reinforcement-learning policy updates, and parameter-efficient fine-tuning to improve predictive performance over time;
compute a numerical risk score for at least one critical illness using outputs of the LLM and the machine-learning classifier;
determine a health risk level for the user by comparing the numerical risk score with a personalized threshold; and
transmit at least one output that includes a notification, a recommendation, and a preventive action to a computing device of the user via the network,
wherein the operations of the artificial-intelligence module improve the functioning of the apparatus by reducing inference latency via cache-augmented generation, decreasing compute-cycle consumption via parameter-efficient fine-tuning, lowering bandwidth usage by transmitting summary-level insights instead of raw data, and reducing false alerts through adaptive threshold recalibration based on real-time physiological trends.

2. The apparatus of claim 1, wherein the user-related medical information comprises medical-record, health-plan data, and unstructured or semi-structured medical-record files.

3. The apparatus of claim 1, wherein the plurality of external data sources includes at least one of an electronic-medical-record system, a personal device, and a payer database.

4. The apparatus of claim 1, wherein the artificial-intelligence module is configured to operate the LLM using:
a cache-augmented generation model configured to merge pre-trained medical data with real-time data from the plurality of external data sources;
a continuous-learning model configured to perform incremental model tuning, which includes at least one of low-rank adaptation, and parameter-efficient fine-tuning;
a self-attention model configured to prioritize clinically significant physiological measurements and diagnostic-code features; and
multimodal embedding alignment configured to correlate physiological-signal vectors with textual medical-record tokens.

5. The apparatus of claim 1, wherein the machine-learning classifier comprises a k-nearest-neighbors (KNN) model configured to compute similarity measures between newly received feature vectors and stored exemplars to produce a discrete health-state classification.

6. The apparatus of claim 1, wherein the personalized threshold is configured to be adaptively updated using at least one of statistical-adjustment techniques, and reinforcement-learning feedback signals derived from user compliance actions and physiological responses.

7. The apparatus of claim 1, wherein the artificial-intelligence module is configured to automatically apply a ruleset mapping the numerical risk score and diagnostic-code clusters to a preventive-care plan that comprises graded intervention levels when the numerical risk score exceeds the personalized threshold.

8. The apparatus of claim 1, wherein the artificial-intelligence module is configured to initiate a remediation sequence that includes scheduled notifications transmitted to the computing device or the wristband until acknowledgment or override when the numerical risk score exceeds the personalized threshold.

9. An apparatus for continuous health monitoring and personalized medical-risk prediction, comprising:
a sensing device integrated in a wearable device, wherein the sensing device is configured to acquire physiological data at predefined intervals;
a wristband having a processor and a memory storing instructions; and
a server comprises an artificial-intelligence module, wherein the artificial-intelligence module comprises a domain-specific large-language model (LLM), a machine-learning classifier, and a data-processing unit, wherein the server is in communication with the wristband,
wherein the server is configured to:
receive the physiological data from the sensing device and receive user-related medical information from a plurality of external data sources, wherein the user-related medical information comprising structured records, unstructured medical narratives, and diagnostic-code data;
standardize the received user-related medical information into time-aligned feature representations and store the standardized data with the physiological data as a unified user state record;
generate, by the data-processing unit, time-dependent structured representations of the physiological data, wherein the time-dependent structured representations comprise temporally aligned feature vectors including statistical summaries, event markers, and contextual annotations;
classify a user health state using the machine-learning classifier by computing similarity metrics between a current physiological feature vector and stored exemplar feature vectors;
supply the classified user health state to the LLM as a structured input token that constrains subsequent inference performed by the LLM;
generate, by the LLM, numeric semantic-relevance scores corresponding to individual physiological feature dimensions, the numeric semantic-relevance scores indicating relative clinical relevance of the physiological feature dimensions;
map the numeric semantic-relevance scores to feature-weight coefficients, and apply the feature-weight coefficients within a similarity computation between the current physiological feature vector and the stored exemplar feature vectors such that modification of the feature-weight coefficients alters a computed classification result of the machine-learning classifier, thereby forming the bidirectional feedback loop in which outputs of the LLM directly influence operation of the machine-learning classifier;
generate predictive insights using the LLM by performing multimodal fusion of physiological features and textual medical-record tokens through a latent-space embedding-alignment layer configured to project both modalities into a shared representation space, cache-augmented generation that retrieves recently ingested medical facts from a dynamically updated memory cache and merges the retrieved facts with pre-trained model parameters to reduce inference latency, and context-aware attention weighting that prioritizes clinically significant physiological measurements and diagnostic-code features;
update personalized thresholds and internal model parameters using a continuous-learning model, wherein the continuous-learning model comprises recency-weighted statistical-adjustment functions applied to the user's historical physiological trends, and reinforcement-learning feedback signals derived from user-compliance actions and resulting physiological responses, the feedback signals being converted into reward values used to update internal policy parameters;
perform parameter-efficient fine-tuning of selected LLM layers using low-rank adaptation or adapter-based modules to enable real-time tuning under wearable-device compute constraints without full model retraining;
determine whether a predictive output exceeds the personalized threshold and, when the condition is satisfied, generate a preventive-care plan mapped from the risk score and diagnostic-code clusters, the preventive-care plan comprising graded intervention levels; and
transmit a set of outputs including a notification, a recommendation, and at least one preventive action to a computing device of the user, wherein the selective transmission of summarized insights in place of raw sensor streams reduces bandwidth consumption and improves real-time responsiveness of the apparatus.

10. The apparatus of claim 9, wherein the user-related medical information comprises medical-record, health-plan data, and unstructured or semi-structured medical-record files.

11. The apparatus of claim 9, wherein the plurality of external data sources includes at least one of an electronic-medical-record system, a personal device, and a payer database.

12. The apparatus of claim 9, wherein the artificial-intelligence module is configured to operate the LLM using a cache-augmented generation model configured to merge pre-trained medical data with real-time data from the plurality of external data sources.

13. The apparatus of claim 9, wherein the server automatically adjusts sensor offsets based on environmental or behavioral context, including ambient temperature, sleep state transitions, and stress-indication signals.

14. The apparatus of claim 9, wherein the artificial-intelligence module is configured to operate the LLM using a self-attention model configured to prioritize clinically significant physiological measurements and diagnostic-code features.

15. The apparatus of claim 9, wherein the reinforcement-learning update loop operates asynchronously with a primary inference pipeline to prevent disruptions to real-time monitoring.

16. The apparatus of claim 9, wherein the artificial-intelligence module is configured to automatically apply a ruleset mapping the numerical risk score and diagnostic-code clusters to a preventive-care plan that comprises graded intervention levels when the numerical risk score exceeds the personalized threshold.

17. The apparatus of claim 9, wherein the artificial-intelligence module is configured to initiate a remediation sequence that includes scheduled notifications transmitted to the computing device or the wristband until acknowledgment or override when the numerical risk score exceeds the personalized threshold.

18. The apparatus of claim 9, wherein the apparatus reduces network bandwidth consumption by generating compressed, classification-level summaries on the server or wearable device instead of transmitting raw sensor streams.

19. The apparatus of claim 9, wherein the feature-weight coefficients applied to modify the similarity metrics are updated at least one of periodically, per inference window, or in response to detection of a physiological anomaly or receipt of new user-related medical information.

20. The apparatus of claim 19, wherein the feature-weight coefficients are updated during successive classification operations such that each classification uses a most recently generated set of feature-weight coefficients.

21. The apparatus of claim 9, wherein the machine-learning classifier comprises a k-nearest-neighbors (KNN) model that computes the similarity metrics using weighted distances between physiological feature vectors.

22. The apparatus of claim 9, wherein the server is configured to provide user-authorized access to health-related outputs through a dedicated application or web-based interface executed on a computing device of an authorized care-team member, the health-related outputs comprising at least one of numerical risk scores, predictive insights, and historical physiological data.

23. The apparatus of claim 22, wherein access to the health-related outputs is role-based and configurable by the user, such that multiple categories of authorized care-team members are permitted to view different subsets of data or receive different alert notifications.

24. The apparatus of claim 9, wherein the server is configured to transmit system-generated alerts or notifications concurrently to the computing device of the user and to the computing device of the authorized care-team member when the numerical risk score exceeds a personalized threshold.

25. A method for adaptive medical-risk prediction using an apparatus for monitoring, tracking, and storing vital signs, comprising:
receiving, by a server, real-time physiological data of a user from a sensing device;
receiving, by a server, user-related medical information from a plurality of external data sources;
standardizing the received user-related medical information into standardized data and generating a user state record;
generating time-dependent structured representations of the physiological data;
classifying a user health state by computing similarity metrics between a current physiological feature vector and stored exemplar feature vectors using a machine-learning classifier;
supplying the classified user health state to a domain-specific large-language model (LLM) as a structured input token that constrains subsequent inference performed by the LLM;
generating, by the LLM, numeric semantic-relevance scores corresponding to individual physiological feature dimensions;
mapping the numeric semantic-relevance scores to feature-weight coefficients, and applying the feature-weight coefficients within a similarity computation between the current physiological feature vector and the stored exemplar feature vectors such that modification of the feature-weight coefficients alters a computed classification result of the machine-learning classifier, thereby forming the bidirectional feedback loop in which outputs of the LLM directly influence operation of the machine-learning classifier;
performing cache-augmented generation to retrieve recently ingested user-specific medical information from a dynamically updated memory cache and inject the retrieved information into an inference context of the LLM;
tuning model parameters using low-rank adaptation or parameter-efficient fine-tuning;
computing a numerical risk score for at least one critical illness using outputs of the LLM and the machine-learning classifier;
updating personalized thresholds using statistical-adjustment or reinforcement-learning feedback;
triggering preventive-care actions and a remediation sequence when the numerical risk score exceeds the personalized thresholds; and
displaying physiological parameters, risk scores, preventive-care options, and remediation outcomes.

26. The method of claim 25, wherein the method further comprises providing, by the server and in response to user authorization, access to health-related outputs through a dedicated application or web-based interface executed on a computing device of an authorized care-team member, the health-related outputs comprising at least one of numerical risk scores, predictive insights, and historical physiological data.

27. The method of claim 25, wherein the method further comprises transmitting system-generated alerts or notifications concurrently to a computing device of the user and to a computing device of the authorized care-team member when the numerical risk score exceeds a personalized threshold.

28. The method of claim 25, wherein the method further comprises enforcing role-based access permissions configurable by the user, such that different categories of authorized care-team members are permitted to access different subsets of the health-related outputs or receive different alert notifications.
